(19) United States
(12) Patent Application Publication
(10) Pub. No.: US 2023/0032607 A1
(43) Pub. Date: Feb. 2, 2023

(54) PROTEIN N-TERMINAL DE NOVO SEQUENCING BY POSITION-SELECTIVE DIMETHYLATION

(71) Applicant: Regeneron Pharmaceuticals, Inc., Tarrytown, NY (US)

(72) Inventors: Yu Feng, Eagleville, PA (US); Shunhai Wang, Scarsdale, NY (US)

(21) Appl. No.: 17/863,349

(22) Filed: Jul. 12, 2022

Related U.S. Application Data
(60) Provisional application No. 63/221,454, filed on Jul. 13, 2021.

(51) Int. Cl. G01N 33/68

(52) U.S. Cl. CPC G01N 33/6824

(57) ABSTRACT

The present invention generally pertains to methods of determining the amino acid sequence of a protein. In particular, the present invention pertains to the use of position-selective dimethylation and liquid chromatography-mass spectrometry to enhance the signal of N-terminal peptides and shift the signal of N-terminal peptides and corresponding b ions, thus facilitating a determination of the sequence of N-terminal peptides.

### CROSS-REFERENCE TO RELATED APPLICATIONS

This application claims priority to and the benefit of U.S. Provisional Patent Application No. 63/221,454, filed Jul. 13, 2021 which is herein incorporated by reference.

### SEQUENCE LISTING

The instant application contains a Sequence Listing which has been submitted electronically in XML format and is hereby incorporated by reference in its entirety. Said XML copy, created on Oct. 24, 2022, is named 070816-02781_SL.xml and is 60,262 bytes in size.

### FIELD

The present invention generally relates to methods for de novo sequencing of proteins.

### BACKGROUND

Protein therapeutics play an important role in the treatment and diagnosis of many diseases. To ensure the integrity and quality of protein therapeutics, it is necessary to determine and confirm protein sequences and other structural properties. A common method for sequencing therapeutic proteins involves the use liquid chromatography-mass spectrometry (LC-MS). However, LC-MS methods have limitations that prevent reliable sequencing of protein N-terminal domains, including low ionization efficiency, ion suppression, and blocking of N-terminal amines.

Various methods have been developed to assist in N-terminal identification, particularly for proteomics applications. Typically they involve chemical modification of amine groups and either positive selection or negative selection to enrich for N-terminal peptides. One such method involves a dimethylation reaction of protein N-terminal residues to assist in identification of the N-terminus. However, these methods are generally applied to identification of proteins in proteomic analysis and not for de novo sequencing of a purified protein. Thus, there exists a need for simple and reliable methods for de novo sequencing of a purified protein.

### SUMMARY

A method has been developed for de novo sequencing of the N-terminal of a protein, as illustrated in FIG. 1. The method includes subjecting a protein in a sample to a position-selective dimethylation reaction such that the N-terminal amine is preferentially dimethylated. The dimethylation reaction may then be quenched with a quenching reagent. The protein may be enzymatically digested and subjected to LC-MS analysis. Dimethylated N-terminal residues form immonium ions which provide a greater signal intensity and a characteristic retention time shift and mass shift, allowing for easy identification of an N-terminal peptide and an N-terminal residue. This identification can then be used to determine the N-terminal sequence of a protein.

This disclosure provides a method for determining an amino acid sequence of an N-terminal domain of a protein of interest. In some exemplary embodiments, the method comprises (a) contacting a sample including a protein of interest to at least one dimethylation reagent to form a dimethylation mixture; (b) contacting said dimethylation mixture to at least one quenching reagent to form a quenched mixture; (c) subjecting said quenched mixture to liquid chromatography-mass spectrometry analysis, wherein said analysis ionizes at least one dimethylated amino acid residue to form at least one immonium ion; (d) identifying at least one N-terminal peptide based on the presence of said at least one immonium ion; and (e) comparing a mass spectrum of said at least one N-terminal peptide of (d) to a mass spectrum of a corresponding at least one N-terminal peptide of a non-dimethylated control sample to determine an amino acid sequence of an N-terminal domain of said protein of interest, wherein said at least one dimethylation reagent of (a) is contacted under conditions that preferentially lead to the dimethylation of an N-terminal α-amine.

In one aspect, said protein of interest is an antibody, a bispecific antibody, a monoclonal antibody, a fusion protein, an antibody-drug conjugate, an antibody fragment, or a protein pharmaceutical product.

In one aspect, said at least one dimethylation reagent is selected from a group consisting of HCHO, NaBH₃CN, heavy isotopes thereof, and a combination thereof. In another aspect, said dimethylation mixture has a pH below 3. In yet another aspect, said dimethylation mixture includes acetic acid. In a further aspect, said dimethylation mixture has a temperature between about 20° C. and about 37° C. In still another aspect, said dimethylation mixture is incubated for between about 5 minutes and about 1 hour.

In one aspect, said quenching reagent is selected from a group consisting of NH₃, NH₂OH, and a combination thereof. In another aspect, said quenched mixture has a temperature between about 20° C. and about 37° C. In yet another aspect, said quenched mixture is incubated for between about 5 minutes and about 1 hour.

In one aspect, the method further comprises contacting said sample and/or said quenched mixture to at least one digestive enzyme. In a specific aspect, said at least one digestive enzyme is selected from a group consisting of trypsin, chymotrypsin, LysC, LysN, AspN, GluC, ArgC, and a combination thereof.

In one aspect, said liquid chromatography comprises reverse phase liquid chromatography, ion exchange chromatography, size exclusion chromatography, affinity chromatography, hydrophobic interaction chromatography, hydrophilic interaction chromatography, mixed-mode chromatography, or a combination thereof. In another aspect, said liquid chromatography system is coupled to said mass spectrometer.

In one aspect, said mass spectrometer is an electrospray ionization mass spectrometer, nano-electrospray ionization mass spectrometer, or a triple quadrupole mass spectrometer. In another aspect, said mass spectrometer is capable performing a multiple reaction monitoring or parallel reaction monitoring.

In one aspect, the method further comprises contacting said sample and/or said quenched mixture to at least one alkylating agent. In a specific aspect, said alkylating agent is iodoacetamide.

In one aspect, the method further comprises contacting said sample and/or said quenched mixture to at least one reducing agent. In a specific aspect, said reducing agent is dithiothreitol.

In one aspect, the method further comprises contact said sample to at least one denaturing agent. In a specific aspect, said denaturing agent is urea.

These, and other, aspects of the present invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 illustrates the state of the art of N-terminal analysis and the need met by the method of the present invention according to an exemplary embodiment.

FIG. 2 illustrates potential N-terminal modifications and C-terminal modifications that affect protein analysis according to an exemplary embodiment.

FIG. 3 shows the structure of an immonium ion generated by collision-induced dissociation (CID) and the amplified signal of said ion in a mass spectrum according to an exemplary embodiment. Figure discloses SEQ ID NOS 9, 9-11 and 10, respectively, in order of appearance.

FIG. 4 illustrates a non-position-selective dimethylation protocol according to an exemplary embodiment. Figure discloses SEQ ID NOS 12-13, respectively, in order of appearance.

FIG. 5 shows sequence coverage of a protein using non-position-selective dimethylation according to an exemplary embodiment. Figure discloses SEQ ID NO: 14.

FIG. 6 shows a mass spectrum including a dimethylated serine immonium ion according to an exemplary embodiment. Figure discloses SEQ ID NOS 41 and 15, respectively, in order of appearance.

FIG. 7 illustrates a position-selective dimethylation protocol according to an exemplary embodiment. Figure discloses SEQ ID NOS 12 and 28, respectively, in order of appearance.

FIG. 8 shows sequence coverage of a protein using position-selective dimethylation according to an exemplary embodiment. Figure discloses SEQ ID NO: 14.

FIG. 9 shows a comparison of total ion chromatograms (TIC) of position-selective dimethylation methods using the molecular weight cut off (MWCO) method or the one-pot method according to an exemplary embodiment.

FIG. 10 shows tested and optimized parameters of the position-selective dimethylation method according to an exemplary embodiment.

FIG. 11A shows a structure of the fusion protein Ab1, including a major truncation species, according to an exemplary embodiment.

FIG. 11B shows an amino acid sequence of Ab1, including major truncation sites, according to an exemplary embodiment. Figure discloses SEQ ID NO: 42.

FIG. 11C shows a mass spectrum of Ab1 analyzed using position-selective dimethylation, with a Y immonium ion of a major truncation site identified, according to an exemplary embodiment. Figure discloses SEQ ID NOS 43 and 16, respectively, in order of appearance.

FIG. 11D shows a mass spectrum of Ab1 analyzed using position-selective dimethylation, with a D immonium ion of a major truncation site identified, according to an exemplary embodiment. Figure discloses SEQ ID NOS 44 and 17, respectively, in order of appearance.

FIG. 11E shows a mass spectrum of Ab1 analyzed using position-selective dimethylation, with a T immonium ion of a major truncation site identified, according to an exemplary embodiment. Figure discloses SEQ ID NOS 45 and 18, respectively, in order of appearance.

FIG. 12 shows a protocol for position-selective dimethylation of NISTmAb and corresponding mass spectra according to an exemplary embodiment. Figure discloses SEQ ID NOS 46,19, 47 and 20, respectively, in order of appearance.

FIG. 13A shows a SEC-MS TIC of FabRICATOR® according to an exemplary embodiment.

FIG. 13B shows a sequence of IdeS according to an exemplary embodiment. Figure discloses SEQ ID NO: 21.

FIG. 13C shows an intact mass spectrum of FabRICATOR® with unknown N-terminal sequences indicated according to an exemplary embodiment. Figure discloses SEQ ID NOS 22-25, respectively, in order of appearance.

FIG. 13D shows mass spectra of FabRICATOR® according to an exemplary embodiment. Figure discloses SEQ ID NOS 1, 27, 1, and 1, respectively, in order of appearance.

FIG. 14A shows chromatograms of control and dimethylated FabRICATOR® N-terminal peptide 1 according to an exemplary embodiment.

FIG. 14B shows MS spectra of control and dimethylated FabRICATOR® N-terminal peptide 1 according to an exemplary embodiment.

FIG. 14C shows MS/MS spectra of control and dimethylated FabRICATOR® N-terminal peptide 1 according to an exemplary embodiment. Figure discloses SEQ ID NOS 2, 29, 2, and 29, respectively, in order of appearance.

FIG. 15A shows chromatograms of control and dimethylated FabRICATOR® N-terminal peptide 2 according to an exemplary embodiment.

FIG. 15B shows MS spectra of control and dimethylated FabRICATOR® N-terminal peptide 2 according to an exemplary embodiment.

FIG. 15C shows MS/MS spectra of control and dimethylated FabRICATOR® N-terminal peptide 2 according to an exemplary embodiment. Figure discloses SEQ ID NOS 3, 30, 3, and 30, respectively, in order of appearance.

FIG. 16A shows chromatograms of control and dimethylated FabRICATOR® N-terminal peptide 3 according to an exemplary embodiment.

FIG. 16B shows MS spectra of control and dimethylated FabRICATOR® N-terminal peptide 3 according to an exemplary embodiment.

FIG. 16C shows MS/MS spectra of control and dimethylated FabRICATOR® N-terminal peptide 3 according to an exemplary embodiment. Figure discloses SEQ ID NOS 4, 31, 4, and 31, respectively, in order of appearance.

FIG. 17A shows chromatograms of control and dimethylated FabRICATOR® N-terminal peptide 4 according to an exemplary embodiment.

FIG. 17B shows MS spectra of control and dimethylated FabRICATOR® N-terminal peptide 4 according to an exemplary embodiment.

FIG. 17C shows MS/MS spectra of control and dimethylated FabRICATOR® N-terminal peptide 4 according to an exemplary embodiment. Figure discloses SEQ ID NOS 5, 32, 5, and 32, respectively, in order of appearance.

FIG. 18A shows chromatograms of control and dimethylated FabRICATOR® N-terminal peptide 5 according to an exemplary embodiment.

FIG. 18B shows MS spectra of control and dimethylated FabRICATOR® N-terminal peptide 5 according to an exemplary embodiment.

FIG. 18C shows MS/MS spectra of control and dimethylated FabRICATOR® N-terminal peptide 5 according to an exemplary embodiment. Figure discloses SEQ ID NOS 6, 33, 6, and 33, respectively, in order of appearance.

FIG. 19A shows chromatograms of control and dimethylated FabRICATOR® N-terminal peptide 6 according to an exemplary embodiment.

FIG. 19B shows MS spectra of control and dimethylated FabRICATOR® N-terminal peptide 6 according to an exemplary embodiment.

FIG. 19C shows MS/MS spectra of control and dimethylated FabRICATOR® N-terminal peptide 6 according to an exemplary embodiment. Figure discloses SEQ ID NOS 26, 34, 26, and 34, respectively, in order of appearance.

FIG. 20A shows an alignment of major FabRICATOR® N-terminal sequences identified using position-selective dimethylation according to an exemplary embodiment. Figure discloses SEQ ID NOS 35, 2-5, 26 and 6, respectively, in order of appearance.

FIG. 20B shows a minor FabRICATOR® N-terminal sequence identified using position-selective dimethylation and corresponding MS/MS spectra according to an exemplary embodiment. Figure discloses SEQ ID NOS 36 and 36-37, respectively, in order of appearance.

FIG. 20C shows FabRICATOR® sequences completed with the major and minor N-terminal sequences identified using position-selective dimethylation according to an exemplary embodiment. Figure discloses SEQ ID NOS 38 and 39, respectively, in order of appearance.

FIG. 20D shows an intact mass spectrum of FabRICATOR® validating the N-terminal sequences identified using position-selective dimethylation according to an exemplary embodiment. Figure discloses SEQ ID NOS 26, 36, 2, 3 and 5, respectively, in order of appearance.

FIG. 20E shows sequence coverage of FabRICATOR® in a control, non-dimethylated sample according to an exemplary embodiment. Figure discloses SEQ ID NO: 40.

FIG. 20F shows sequence coverage of FabRICATOR® in a position-selective dimethylated sample according to an exemplary embodiment. Figure discloses SEQ ID NO: 40.

FIG. 21A shows optimized conditions for position-selective dimethylation according to an exemplary embodiment.

FIG. 21B illustrates a method for immonium ion-triggered MS/MS data acquisition according to an exemplary embodiment. Figure discloses SEQ ID NO: 31.

### DETAILED DESCRIPTION

Protein therapeutics, especially monoclonal antibodies, play a significant role in the treatment and diagnosis of many diseases. Poor therapeutic protein quality can cause undesired immunogenic responses in patients, loss of drug potency, or adverse effects. To ensure the integrity and quality of protein therapeutics, it is necessary to determine and confirm protein sequences and other structural properties.

A common method for analysis of therapeutic proteins, including sequencing, involves the use of liquid chromatography-mass spectrometry. A peptide sequence may be assigned from the analysis of MS/MS fragments obtained from collision-induced dissociation (CID) or post-source decay (PSD) of a selected molecular ion. However, identification of the N-terminal peptide of a protein presents unique challenges. b ions observed in CID mass spectra typically form stable structures by cyclization of protonated oxalozone molecules. However, this cyclization is not possible for the b₁ ion, comprising the N-terminal residue of an N-terminal peptide, leading to an omission of the b₁ ion in mass spectra and an inability to determine the N-terminal residue of a protein with conventional methods (Hsu et al., 2005, J Proteome Res, 4:101-108).

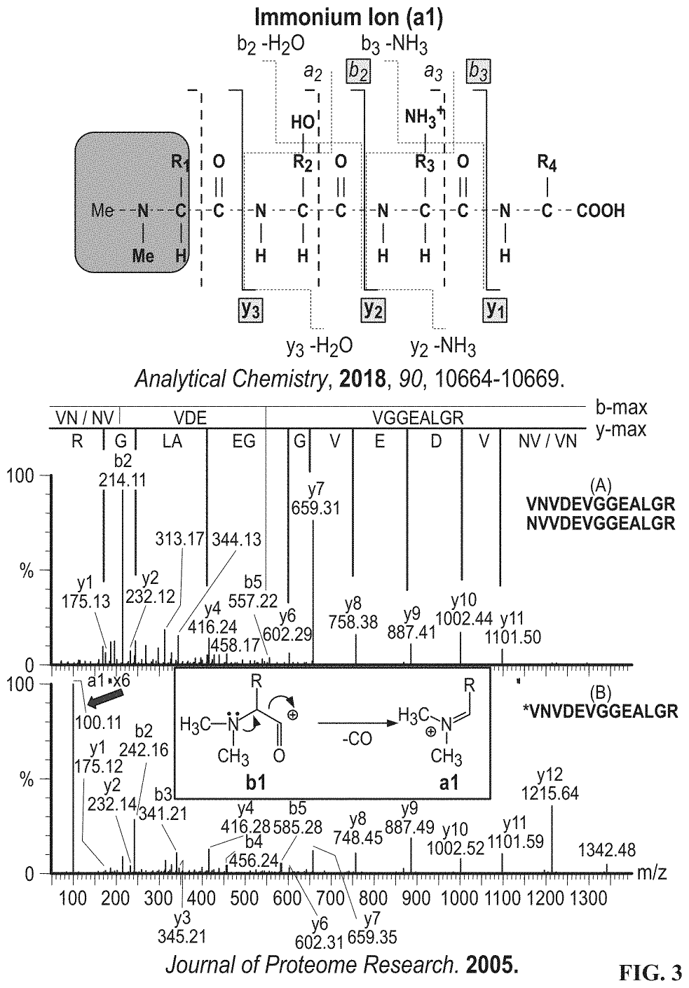

A number of methods have been developed to assist in N-terminal identification, particularly for proteomics applications. Typically they involve chemical modification of amine groups and either positive selection or negative selection to enrich for N-terminal peptides (Niedermaier et al., 2019, Biochim Biophys Acta Proteins Proteom, 1867(12):140138). A particular method involves the use of formaldehyde to cause dimethylation of an N-terminal α-amine group and lysine ε-amine groups (Hsu et al.). A dimethylated N-terminal residue forms an immonium ion when ionized, enhancing its ionization efficiency and detectable signal in MS, as shown in FIG. 3. N-terminal dimethylation also causes a predictable mass shift that allows the N-terminal peptide and b ions comprising the N-terminal residue to be easily identified.

Dimethylation techniques for proteomics have been further optimized, for example with the TAILS technique or DiLeu cPILOT technique (Marino et al., 2015, ACS Chem Biol, 10:1754-1764; Frost et al., 2018, Anal Chem, 90:10664-10669). Frost et al. demonstrated the use of acidic conditions to modify a dimethylation reaction: by performing the reaction at a low pH, N-terminal α-amine groups (which have a lower pKa) preferentially react while lysine side chain ε-amine groups (which have a higher pKa) preferentially remain unmodified. Light isotopic and heavy isotopic dimethylation reagents were used to create dimethylation samples of contrasting masses. This method was combined with isobaric tagging of lysines to perform 24-plex proteomics analysis of a complex sample to identify proteins in the sample. However, this and other described N-terminal labeling methods have typically been restricted to use in proteomics, and have not been applied to de novo sequencing of purified proteins, as is needed for example to characterize therapeutic proteins for drug development.

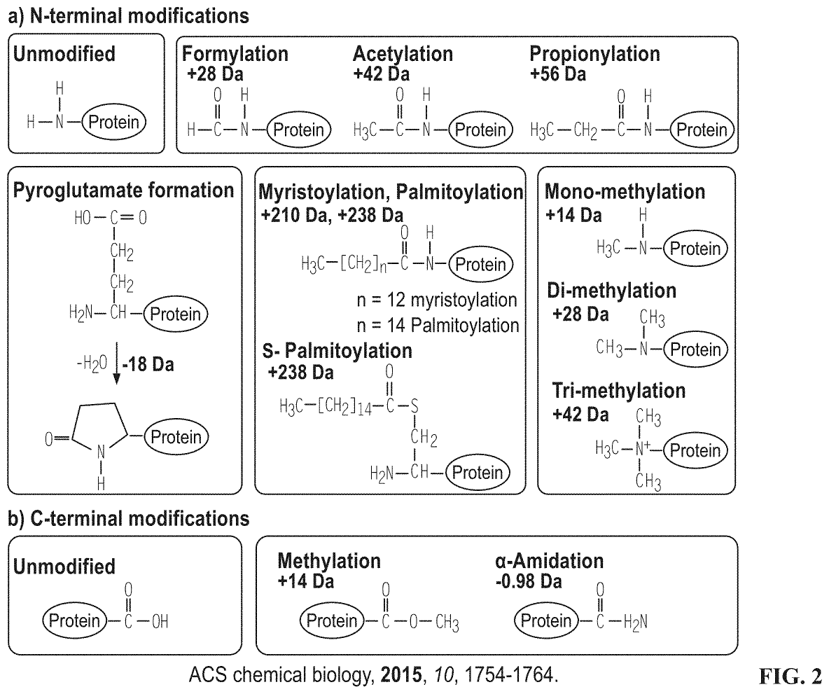

More recently, a method was developed for de novo N-terminal sequencing of a purified protein by fluorescently labeling unblocked N-terminal residues (Vecchi et al., 2019, Anal Chem, 91:13591-13600). This method requires the use of an online fluorescence detector, and was not capable of labeling N-terminals that were predominantly blocked, for example with pyroglutamate. Vecchi et al. attempted to circumvent this issue by adding a second experimental track comparing samples that were digested with pyroglutamate aminopeptidase (PGAP), removing the pyroQ residue, to undigested samples. This workaround of the inability of the labeling process to sufficiently identify N-terminal peptides adds a layer of complexity and cannot account for any N-terminal modifications besides pyroQ, for example the modifications illustrated in FIG. 2.

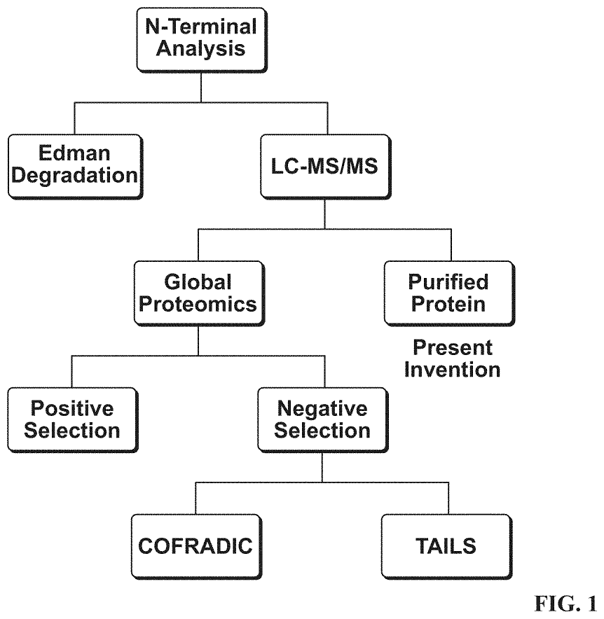

As described above and illustrated in FIG. 1, there exists a need for simple and sensitive methods for de novo sequencing of purified proteins, particularly for the challenging N-terminal domain. This disclosure sets forth a novel method of labeling, identifying and de novo sequencing the N-terminal domain of a protein.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein of interest can be a recombinant protein, an antibody, a bispecific antibody, a multispecific antibody, antibody fragment, monoclonal antibody, fusion protein, scFv and combinations thereof.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 (or “Fab₂”) fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. In some exemplary embodiments, an antibody fragment may be produced by digestion with the digestive enzyme IdeS or a variant thereof. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications, which are hereby incorporated by reference: U.S. Ser. No. 12/823,838, filed Jun. 25, 2010; U.S. Ser. No. 13/488,628, filed Jun. 5, 2012; U.S. Ser. No. 14/031,075, filed Sep. 19, 2013; U.S. Ser. No. 14/808,171, filed Jul. 24, 2015; U.S. Ser. No. 15/713,574, filed Sep. 22, 2017; U.S. Ser. No. 15/713,569, field Sep. 22, 2017; U.S. Ser. No. 15/386,453, filed Dec. 21, 2016; U.S. Ser. No. 15/386,443, filed Dec. 21, 2016; U.S. Ser. No. 15/22343 filed Jul. 29, 2016; and U.S. Ser. No. 15/814,095, filed Nov. 15, 2017.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

In some exemplary embodiments, the protein of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

As used herein, “sample” can be obtained from any step of the bioprocess, such as cell culture fluid (CCF), harvested cell culture fluid (HCCF), any step in the downstream processing, drug substance (DS), or a drug product (DP) comprising the final formulated product. In some other specific exemplary embodiments, the sample can be selected from any step of the downstream process of clarification, chromatographic production, viral inactivation, or filtration. In some specific exemplary embodiments, the drug product can be selected from manufactured drug product in the clinic, shipping, storage, or handling.

In some exemplary embodiments, a protein of interest may be prepared by, for example, alkylation, reduction, denaturation, and/or digestion.

As used herein, the term “protein alkylating agent” refers to an agent used for alkylating certain free amino acid residues in a protein. Non-limiting examples of protein alkylating agents are iodoacetamide (IAA), chloroacetamide (CAA), acrylamide (AA), N-ethylmaleimide (NEM), methyl methanethiosulfonate (MMTS), and 4-vinylpyridine or combinations thereof. In an exemplary embodiment, iodoacetamide is used as an alkylating agent.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof. In an exemplary embodiment, urea is used as a denaturing agent.

As used herein, the term “protein reducing agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof. In an exemplary embodiment, DTT is used as a reducing agent.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)). In an exemplary embodiment, trypsin and LysC are used as digestive enzymes.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application. In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on, as long as one can obtain meaningful information, or the fragment ion signal is detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device. The peptides identified by the mass spectrometer can be used as surrogate representatives of the intact protein and their post translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

In some exemplary aspects, the mass spectrometer can work using nanoelectrospray or nanospray.

The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary aspects, the mass spectrometer can be a tandem mass spectrometer.

As used herein, the term “tandem mass spectrometry” includes a technique where structural information on sample molecules is obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on as long as one can obtain meaningful information, or the fragment ion signal is detectable. Tandem MS has been successfully performed with a wide variety of analyzer combinations. What analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post-translational modifications, or comparability analysis, or combinations thereof.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsfedu/prospector/mshome.htm), Byonic (www.proteinmetrics.com/products/byonic) or Sequest (fields.scripps.edu/sequest).

In some exemplary embodiments, the mass spectrometer is coupled to the liquid chromatography system.

In some exemplary embodiments, the mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring—based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

In some aspects, the mass spectrometer in the method or system of the present application can be an electrospray ionization mass spectrometer, nano-electrospray ionization mass spectrometer, or a triple quadrupole mass spectrometer, wherein the mass spectrometer can be coupled to a liquid chromatography system, wherein the mass spectrometer is capable of performing LC-MS (liquid chromatography-mass spectrometry) or LC-MRM-MS (liquid chromatography-multiple reaction monitoring-mass spectrometry) analyses.

As used herein, the term “mass analyzer” includes a device that can separate species, that is, atoms, molecules, or clusters, according to their mass. Non-limiting examples of mass analyzers that could be employed are time-of-flight (TOF), magnetic electric sector, quadrupole mass filter (Q), quadrupole ion trap (QIT), orbitrap, Fourier transform ion cyclotron resonance (FTICR), and also the technique of accelerator mass spectrometry (AMS).

It is understood that the present invention is not limited to any of the aforesaid protein(s) of interest, antibody(s), sample(s), liquid chromatography method(s) or system(s), mass spectrometer(s), alkylating agent(s), reducing agent(s), digestive enzyme(s), database(s), or bioinformatics tool(s), and any protein(s) of interest, antibody(s), sample(s), liquid chromatography method(s) or system(s), mass spectrometer(s), alkylating agent(s), reducing agent(s), digestive enzyme(s), database(s), or bioinformatics tool(s) can be selected by any suitable means.

The present invention will be more fully understood by reference to the following Examples. They should not, however, be construed as limiting the scope of the invention.

### EXAMPLES

Position-selective one pot dimethylation protocol. A protocol for position-selective one pot dimethylation is described herein. 100 μg of purified protein was obtained. The protein was denatured in 10 μL (10 μg/μL) 8 M urea at 50° C. for 10 minutes. The sample was cooled down. A dimethylation reaction mixture was added comprising 2.5 μL 8 M urea containing 5% acetic acid, 300 mM HCHO and 120 mM NaBH₃CN, and the reaction was allowed to proceed for 15 minutes at 37° C. 2.5 μL of 8 M urea containing 2.5% NH₂OH was added to quench the dimethylation reaction, and incubated for 15 minutes at 37° C.

The protein was then reduced by adding 2.5 μL 8 M urea in 0.4 M Tris pH 7.5 with 20 mM dithiothreitol (DTT), and incubated at 37° C. for 15 minutes. The protein was alkylated and digested by the addition of 2.5 μL 125 mM iodoacetamide (IAA) and 2 μL 0.5 μg/μL rLys-C (substrate to enzyme ratio of 100), and incubated in the dark at 37° C. for 15 minutes. Afterwards 160 μL 0.1 M Tris pH 7.5 was added to dilute the sample and 10 μL 0.5 μg/μL trypsin (substrate to enzyme ratio of 20) was added for additional digestion, and incubated at 37° C. for 2 hours. 3.5 μL of 5.75 mU/μL PNGase F was added to each sample (substrate to enzyme ratio of 5, by weight), and incubated at 37° C. for 1 hour. Finally 2 μL of 10% formic acid (FA) was added to stop digestion, before LC-MS analysis.

Further optimized protocol. A further optimized protocol for position-selective one pot dimethylation was developed. 200 μg of purified protein was obtained. The protein was denatured and reduced in 20 μL (10 μg/μL) 8 M urea with 5 mM DTT at 37° C. for 30 minutes. The protein was alkylated by adding 2.5 μL 8 M urea containing 125 mM IAA and incubated in the dark at 37° C. for 15 minutes. A dimethylation reaction mixture was added comprising 2.5 μL 8 M urea containing 10% acetic acid, 600 mM HCHO and 240 mM NaBH₃CN, and the reaction was allowed to proceed for 30 minutes at 37° C. 5 μL of 8 M urea containing 2.5% NH₂OH was added to quench the dimethylation reaction, and incubated for 30 minutes at 37° C.

340 μL 0.1 M Tris pH 7.5 was added to dilute the sample and 20 μL 0.5 μg/μL trypsin (substrate to enzyme ratio of 20) was added for digestion, and incubated at 37° C. for 2 hours. 7 μL of 5.75 mU/μL PNGase F was added to each sample (substrate to enzyme ratio of 5, by weight), and incubated at 37° C. for 1 hour. Finally 4 μL of 10% FA was added to stop digestion, before LC-MS analysis.

### Example 1. Non-Position-Selective, Molecular Weight Cut Off Method

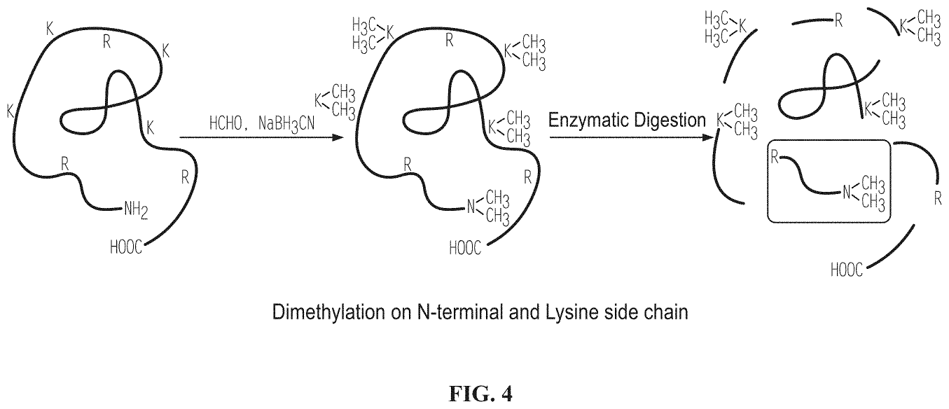

A new method for de novo sequencing of purified proteins was developed using dimethylation sample preparation and LC-MS analysis. In order to optimize the conditions of the method, a variety of approaches were tested and compared. An initial approach was tested as illustrated in FIG. 4. In this approach, an intact protein is treated with dimethylation reagents in a non-position-selective manner, leading to dimethylation of the N-terminal α-amine group as well as the ε-amine group of lysine side chains, and then dimethylation reagents are removed by buffer exchange with a molecular weight cut off (MWCO) filter.

Specifically, the sample is denatured with urea, and incubated with HCHO and NaBH₃CN to dimethylate amine groups. The sample is subjected to buffer exchange with a 30K MWCO filter to remove the dimethylation reagents. The sample is then subjected to cysteine reduction using dithiothreitol (DTT) and alkylation with iodoacetamide (IAA). The protein is subjected to enzymatic digestion with rLys-C and trypsin, and finally subjected to LC-MS analysis.

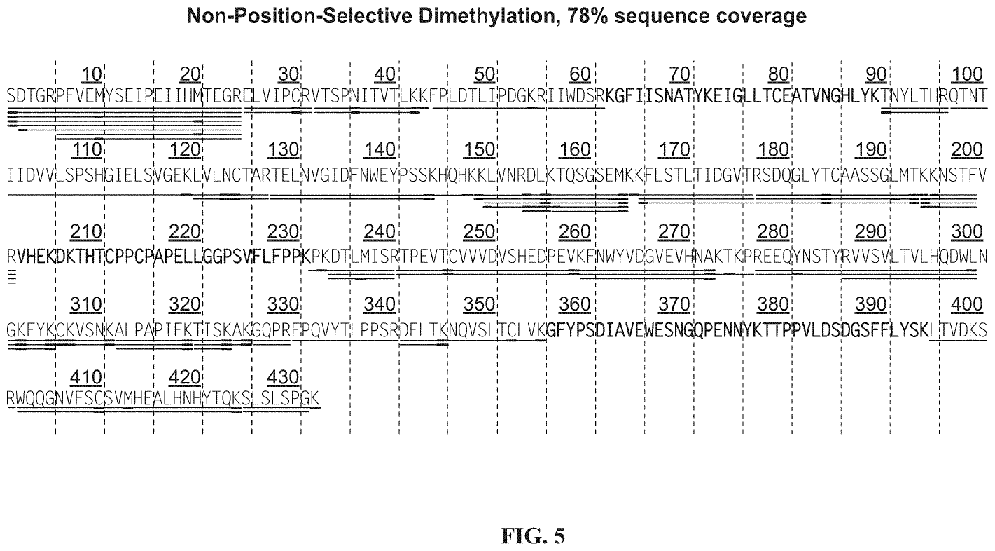

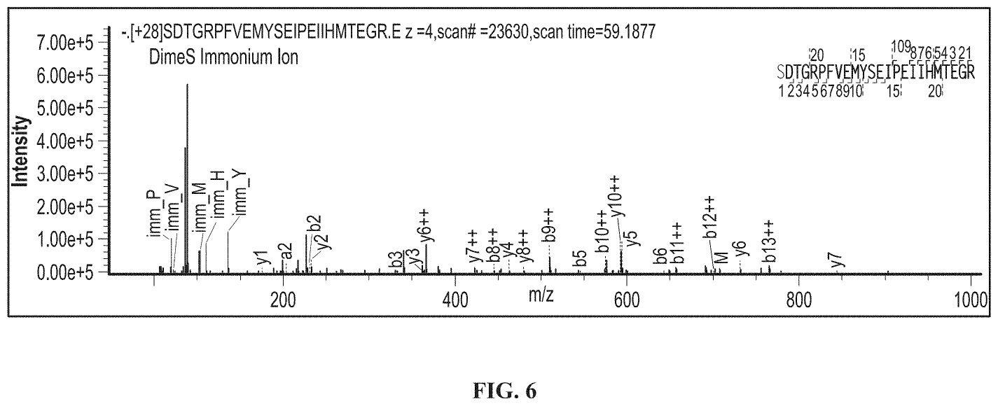

Exemplary results using this method for a known protein sequence are shown in FIG. 5. Over 95% yield was achieved for dimethylation of the N-terminal serine (S) for an exemplary protein sequence. 78% sequence coverage was achieved. As shown in the mass spectrum of FIG. 6, enhanced dimethylated immonium ion was clearly observed after higher-energy C-trap dissociation (HCD) fragmentation.

Potential drawbacks of the method included that non-specific modification of the ε-amine of lysine can interfere with enzymatic digestion, leading to the generation of longer sequences and lower sequence coverage. Additionally, buffer exchange by MWCO adds a considerable amount of time to carry out the method, and causes sample loss, potentially leading to a lower signal in the total ion chromatogram (TIC).

### Example 2. Position-Selective, Molecular Weight Cut Off Method

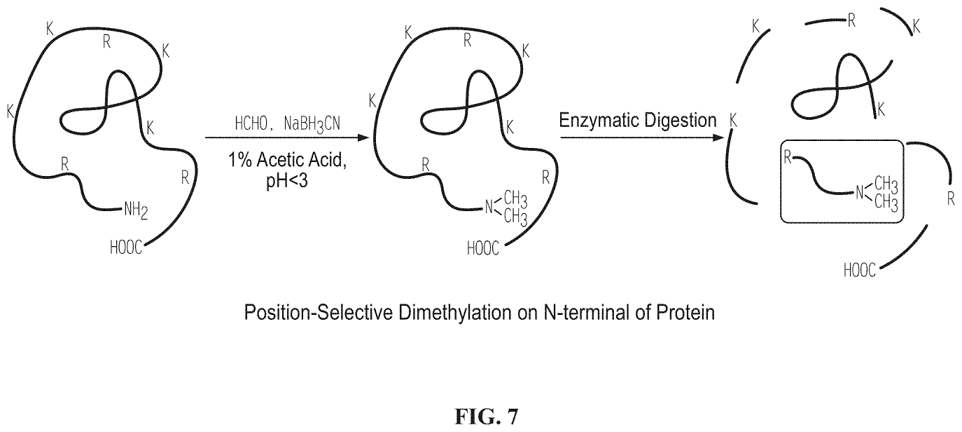

In order to improve detection and assist analysis of the N-terminus of a protein, the method described in Example 1 was further modified. Instead of employing non-position selective dimethylation of amines, position-selective dimethylation was used, as shown in FIG. 7. Because of the difference in pKa of the α-amine group of the N-terminus compared to the ε-amine group of lysine side chains (roughly 8 and 10 respectively), each will preferentially chemically react at a different pH. Thus, by controlling the pH of the dimethylation reaction using the addition of 1% acetic acid, particularly to achieve a pH below 3, the N-terminal amine can be preferentially dimethylated while lysines remain relatively unmodified.

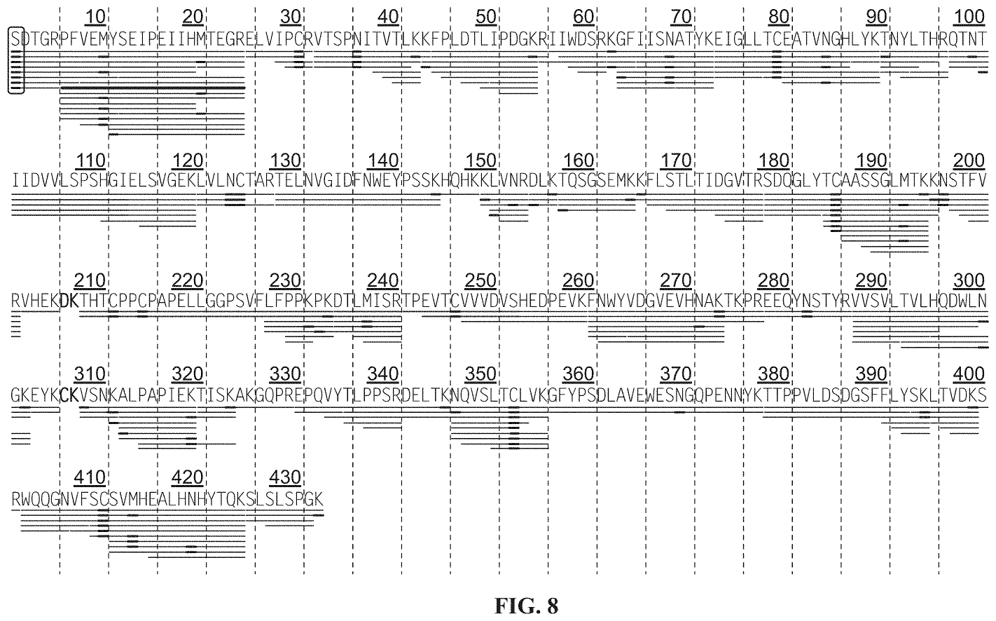

Exemplary results using this method for a known protein sequence are shown in FIG. 8. Analysis showed that over 99% yield was achieved for N-terminal dimethylation, while less than 0.1% dimethylation was observed at the ε-amine of lysines and internal peptides. Thus, position-selective dimethylation allowed for a considerable improvement in the detection of an N-terminal peptide.

### Example 3. Position-Selective, One-Pot Method

In order to increase the signal achievable with LC-MS and further improve identification and sequencing of N-terminal peptides, the method of Example 2 was further modified. The buffer exchange with MWCO step was replaced with a quenching step, using the addition of NH₂OH to the mixture after the dimethylation step to prevent further dimethylation reactions. The omission of a buffer exchange step allowed for reduced loss of sample and thus higher signal intensity.

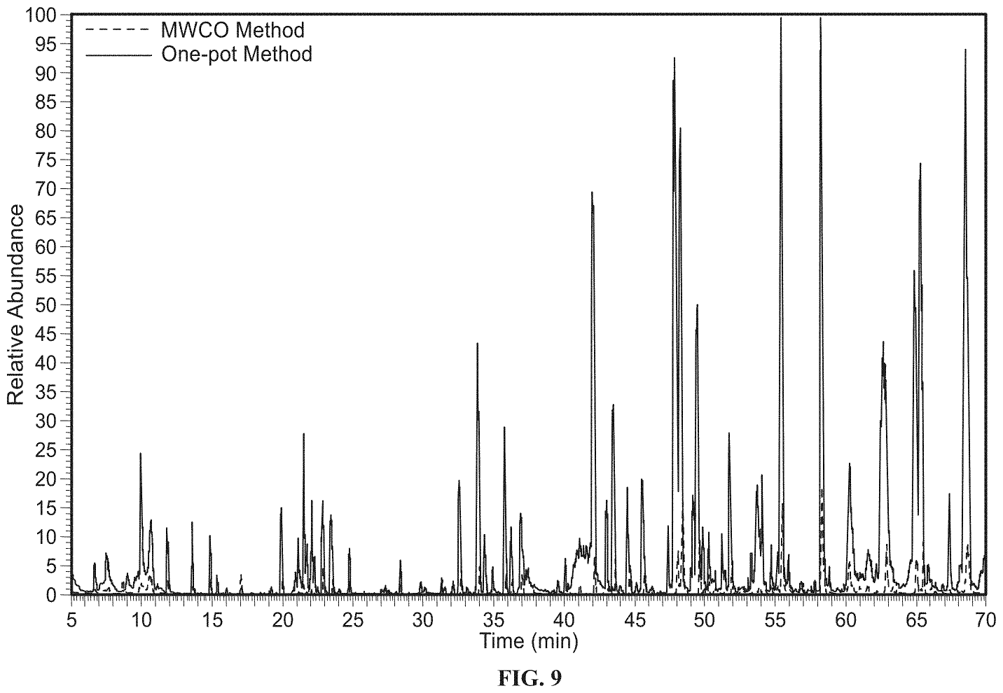

Exemplary results using this method, compared to the MWCO method of Example 2, are shown in FIG. 9. As with the previous method, high yield was achieved for N-terminal dimethylation, and less than 0.1% dimethylation was observed at the ε-amine of lysines and internal peptides. However, this one-pot method showed a dramatic improvement in TIC signal compared to the MWCO method, allowing for more effective detection and sequencing of N-terminal peptides.

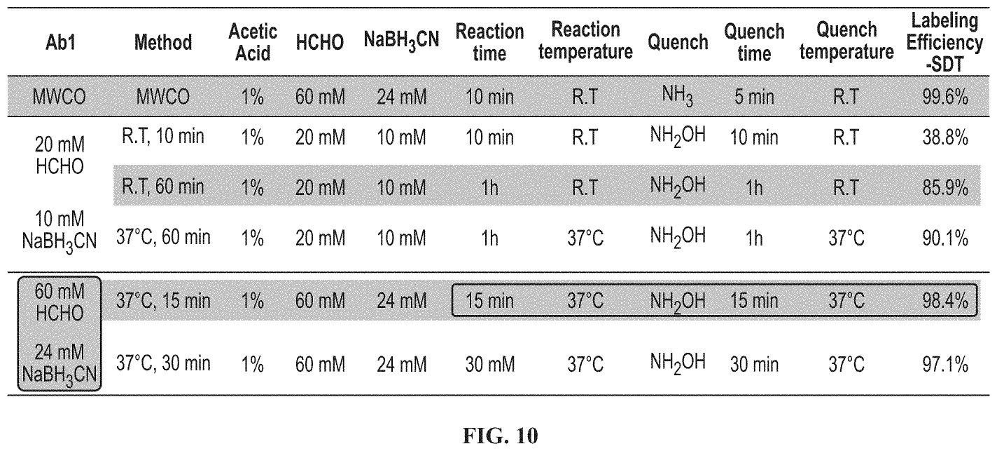

These and other parameters were optimized for the method of the invention, as shown in FIG. 10 and described in detail under “Position-selective one pot dimethylation protocol” above. The optimal parameters selected for future experiments included the use of 8 M urea to initially denature the protein. For the dimethylation reaction, the optimal parameters selected included the use of 1% acetic acid, 60 mM HCHO, 24 mM NaBH₃CN, a reaction time of 15 minutes, and a reaction temperature of 37° C. For the quenching process, the optimal parameters selected included the use of NH₂OH, for 15 minutes, at 37° C. Finally, DTT was selected as a reducing agent and iodoacetamide as an alkylating agent.

### Example 4. Method Validation of Position-Selective Dimethylation with Known Protein Sequences

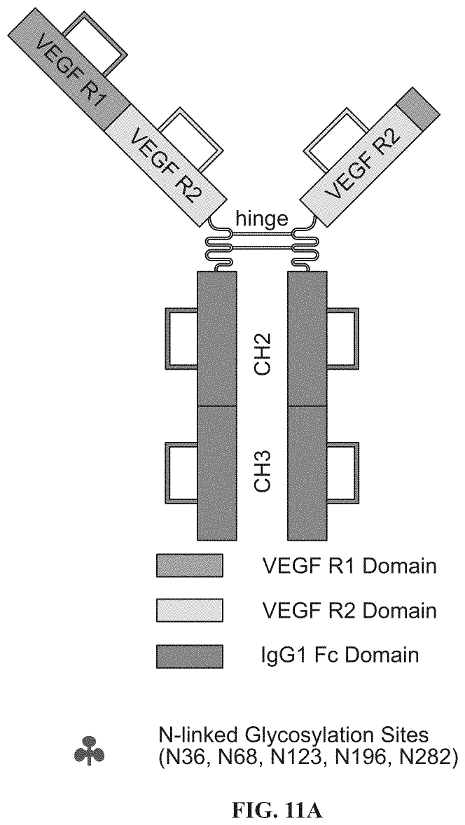

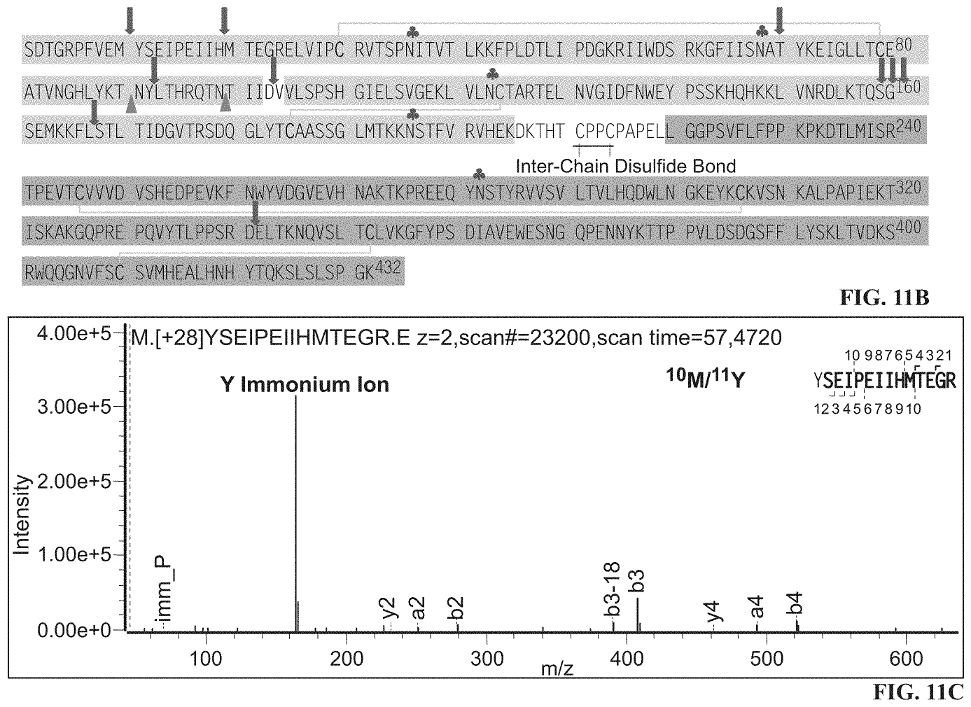

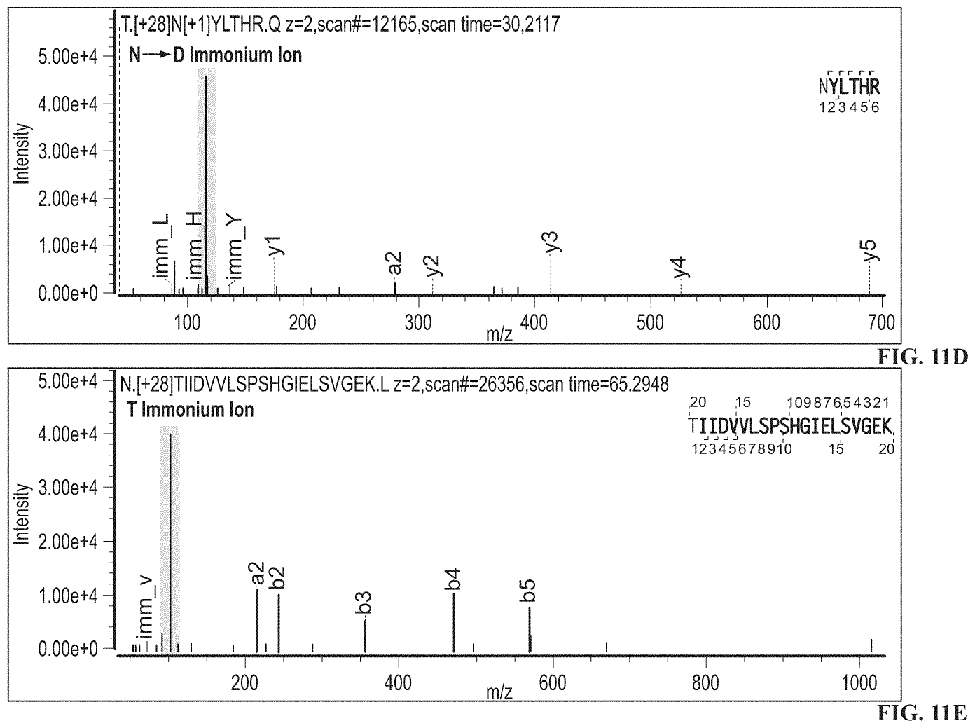

In order to validate the use of the method of the present invention, proteins with known sequences were subjected to de novo N-terminal sequencing using position-selective dimethylation. FIG. 11A illustrates the structure of an antibody fusion protein, Ab1. Ab1 features major truncation species, leading to a heterogeneity of N-termini. FIG. 11B illustrates a sequence of Ab1, including arrows indicating major truncation sites, for example at ¹⁰M/¹¹Y, ⁹⁰T/⁹¹N, and ⁹⁹N/¹⁰⁰T.

Ab1 was subjected to de novo N-terminal sequencing by position-selective dimethylation, and N-termini produced by truncation were successfully detected using the method of the present invention. FIG. 11C shows detection of the Y immonium ion derived from the ¹⁰M/¹¹Y truncated protein. FIG. 11D shows detection of the D immonium ion derived from the ⁹⁰T/⁹¹N truncation. FIG. 11E shows detection of the T immonium ion derived from the ⁹⁹N/¹⁰⁰T truncation.

Notably, ⁹⁹N/¹⁰⁰T is also a site of non-specific trypsin cleavage. Because the dimethylation reaction occurs and is then quenched before digestion, only N-terminal amines present before digestion are dimethylated and produce immonium ions, allowing for the differentiation of peptide fragments with the same amino acid sequence that were derived from in vivo truncation compared to experimental digestion.

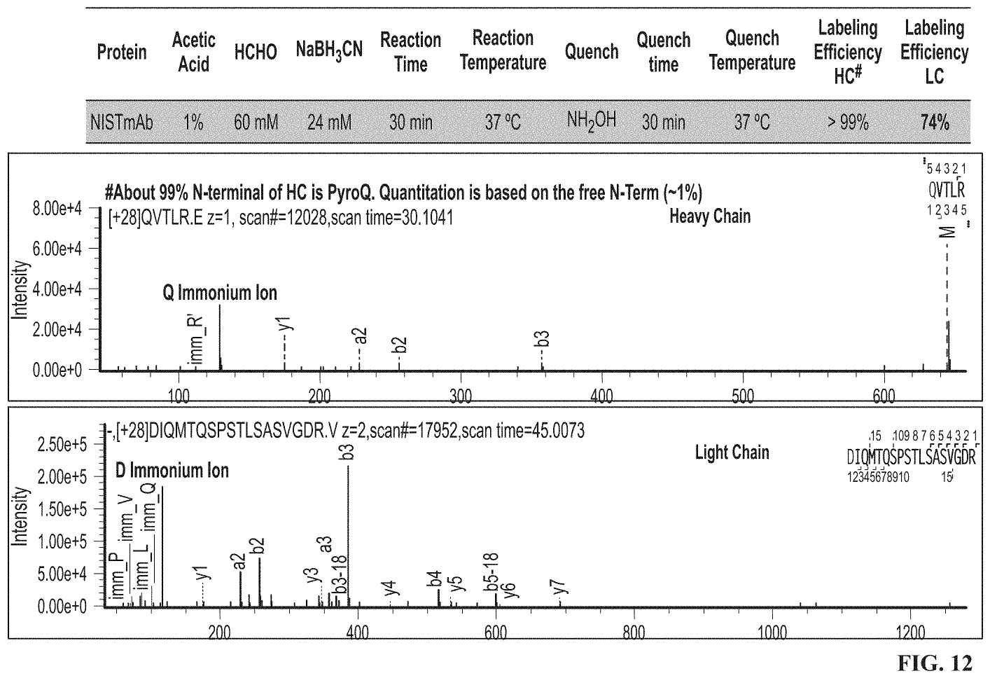

The method of the present invention was further validated using another protein with a known sequence: the monoclonal antibody standard NISTmAb. Roughly 99% of the N-terminal of the NISTmAb heavy chain (HC) is blocked by pyroglutamate (pyroQ), preventing participation in the dimethylation reaction. Blocking of the N-terminal, by pyroQ or any of a number of other modifications, is a common challenge for techniques that rely on modification of the free N-terminal amine. However, the method of the present invention demonstrates high enough sensitivity that the N-terminal peptide may be identified even with the vast majority of the N-terminus blocked. Exemplary methods and results of the analysis of NISTmAb are shown in FIG. 12, showing successful identification of the Q immonium ion of the heavy chain and D immonium ion of the light chain despite the blocked N-terminal.

### Example 5. Case Study of Unknown Protein N-Terminal De Novo Sequencing

The method of the present invention was used for de novo sequencing of an unknown protein N-terminal, demonstrating its utility in real-world application.

The IdeS protease, derived from Streptococcus pyogenes, is a valuable tool in the development of antibody therapeutics (U.S. Publication Number 2007/0237784 A1). IdeS specifically cleaves an IgG antibody below the hinge region, generating two Fc/2 fragments and one F(ab′)₂ (or Fab₂) fragment. A recombinantly modified form of IdeS featuring a His tag is commercially available from Genovis under the name of FaRICATOR®.

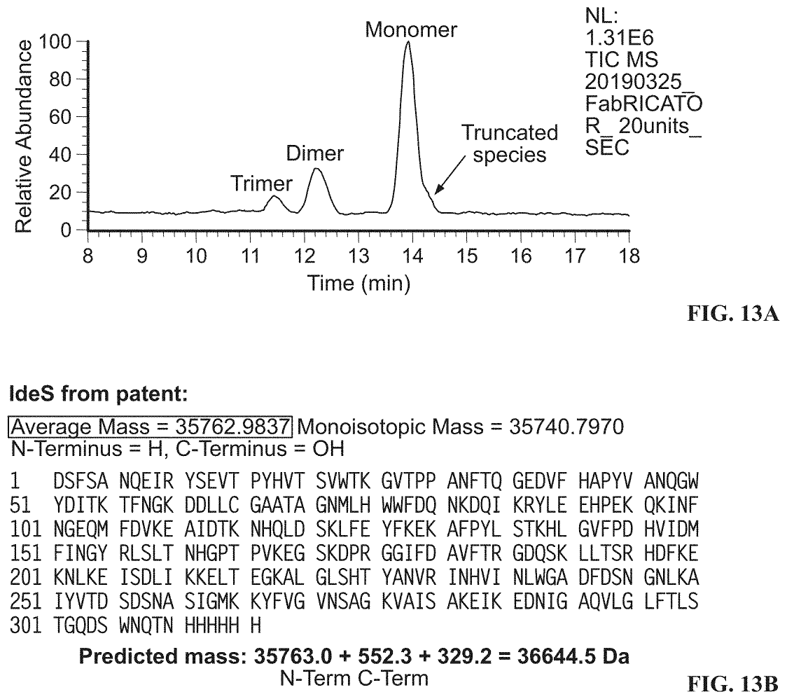

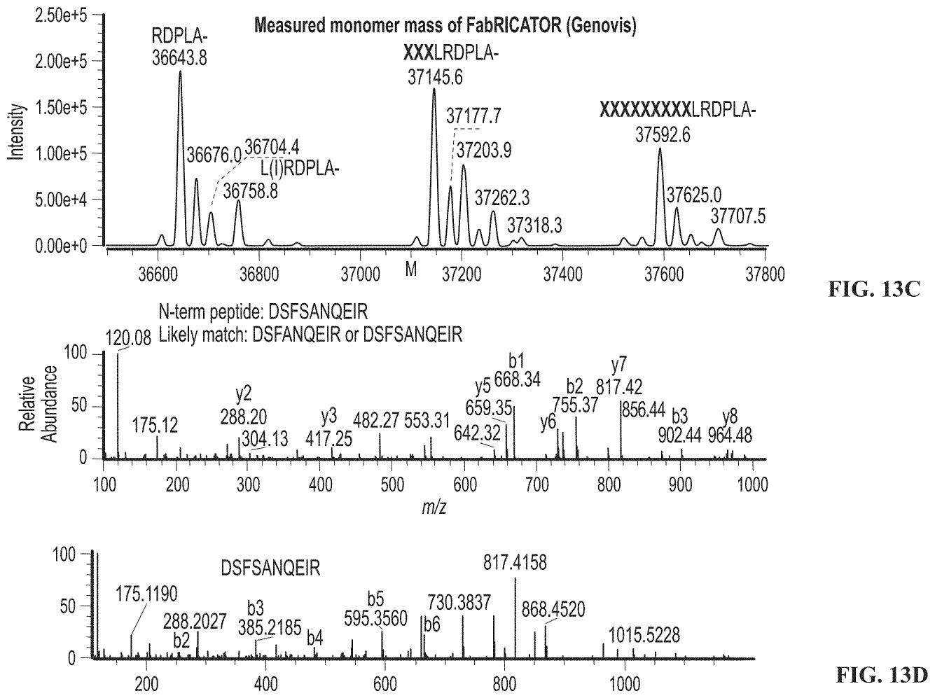

A TIC from intact SEC-MS analysis of FabRICATOR® is shown FIG. 13A, demonstrating that in addition to a main monomer species, FabRICATOR® comprises a trimer, dimer, and uncharacterized truncated species. Genovis describes FabRICATOR® as having a molecular weight of 37,725 Da. In contrast, the predicted mass of the originally published IdeS sequence is 36,644.5 Da, as shown in FIG. 13B. This suggests that FabRICATOR® comprises additional, undisclosed amino acids compared to IdeS, truncations of which could potentially give rise to the truncated species seen by SEC-MS. Mass spectra from intact mass analysis and peptide mapping analysis of FabRICATOR® are shown in FIG. 13C and 13D respectively. Conventional mass spectrometry methods were unable to identify the N-terminal sequence of FabRICATOR®. Undisclosed potential N-terminal sequences prior to the disclosed IdeS N-terminal sequence of DSFSANQEIR (SEQ ID NO: 1) are indicated.

In order to conduct de novo sequencing of an unknown N-terminal sequence, a control sample and a dimethylated sample were prepared in parallel. The total amount of FabRICATOR® in each starting sample was 10 μg (0.05 μg/μL). Both samples were prepared and analyzed using the position-selective dimethylation method described above, with the exception that dimethylation reagents were not added to the control sample. The chromatographic injection amount for the protein in each sample was 2 μg/40 μL. Each peak pair of N-terminal sequences was manually identified. The dimethylated peptide was distinguishable by having a slightly increased LC retention time, and a mass increase of 28 Da. For de novo sequencing, each b ion from the control versus dimethylated sample was separated by 28 Da due to the dimethylated N-terminal residue, while each y ion had the same accurate mass, allowing for easy identification of b and y ions, and thus clear and efficient sequencing. The results were then cross-validated using additional techniques including intact MS.

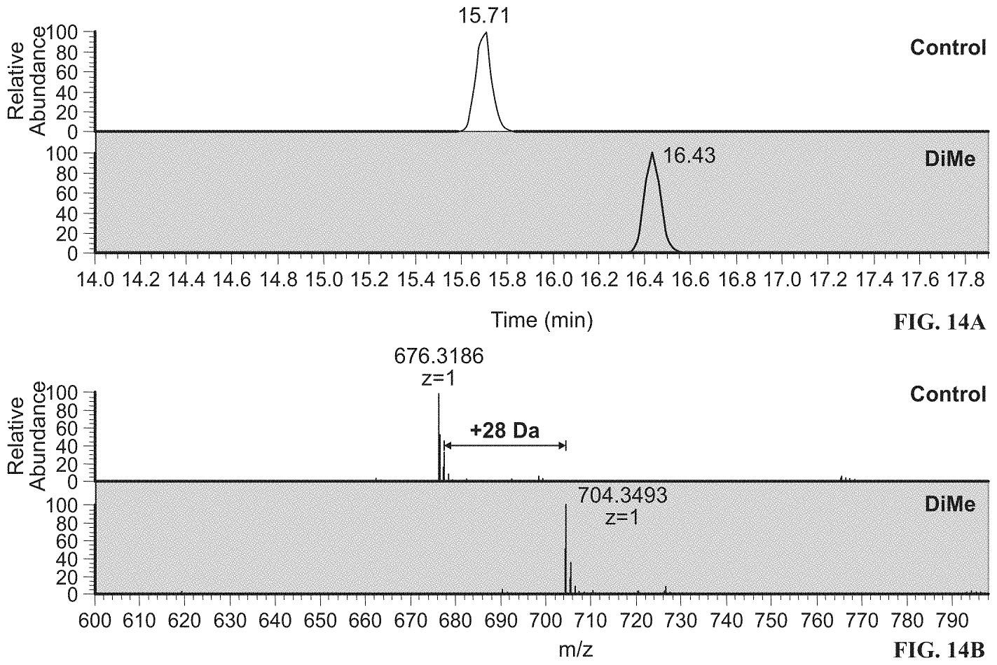

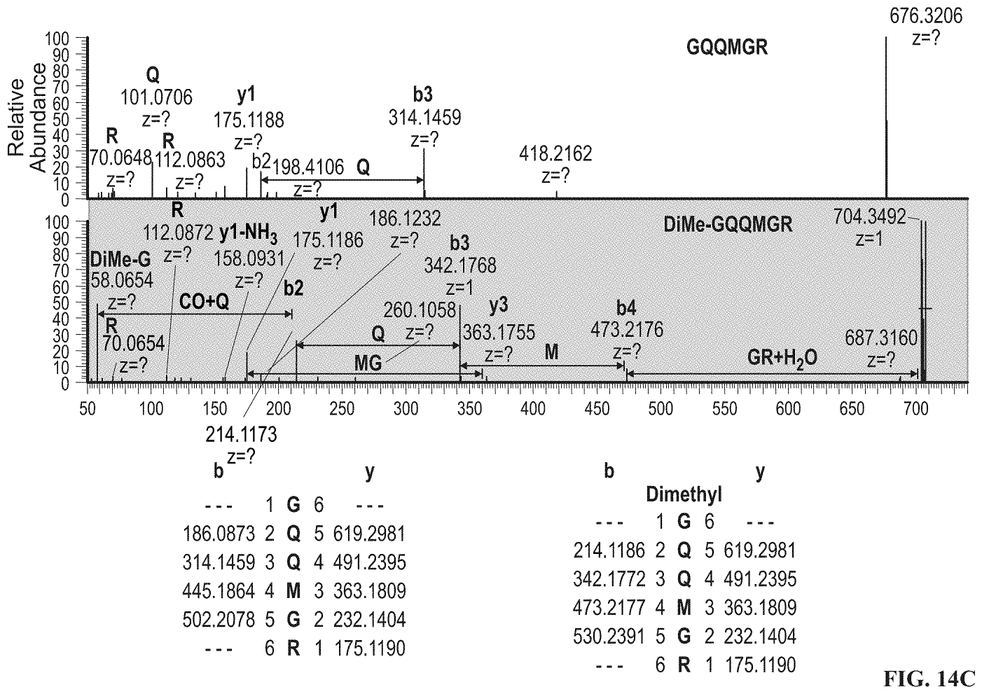

FIG. 14A shows a chromatogram of FabRICATOR® N-terminal peptide 1, comparing the control and dimethylated (DiMe) peptide. The dimethylated peptide shows an increased retention time. FIG. 14B shows a corresponding mass spectrum, showing that the dimethylated N-terminal peptide has the predicted mass shift of 28 Da. FIG. 14C shows an MS/MS spectrum of FabRICATOR® N-terminal peptide 1 from the control sample. The identity of the first amino acid in the sequence is not distinguishable here, and thus sequencing is not possible using conventional LC-MS/MS. In contrast, FIG. 14D shows the corresponding spectrum from the dimethylated sample. Here, the dimethylated G residue is clearly visible as the first amino acid in the sequence. By comparing the spectra of FIG. 14C and 14D, the identity of b ions is clearly distinguishable based on having a 28 Da mass shift, compared to y ions which do not have a mass shift in the dimethylated sample. This is also indicated in the table of b and y ions below each spectrum. Using the method of the present invention, FabRICATOR® N-terminal peptide 1 was identified as having the sequence of GQQMGR (SEQ ID NO: 2).

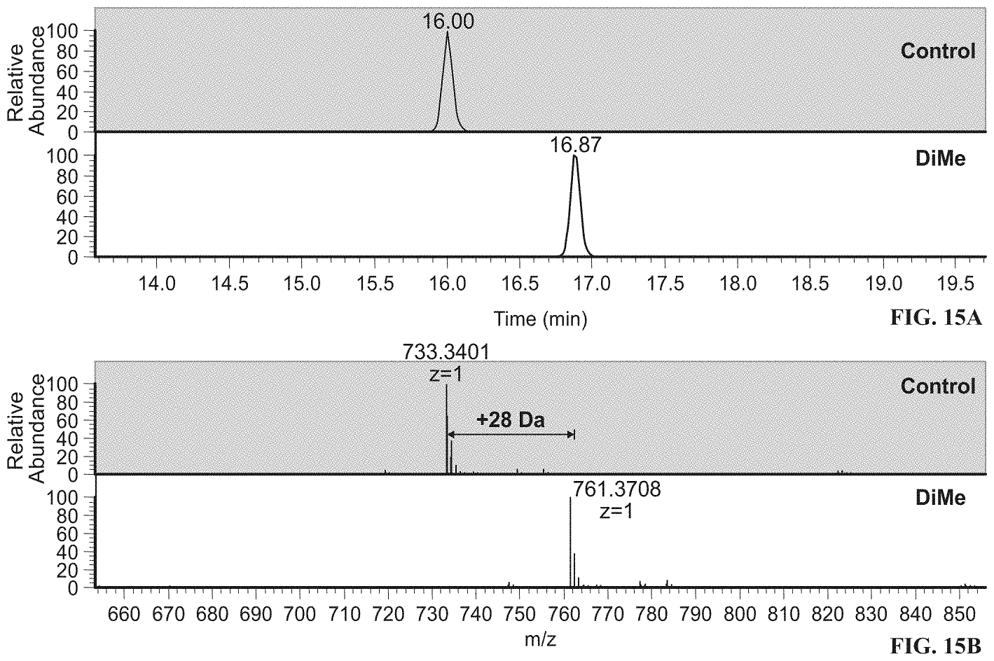

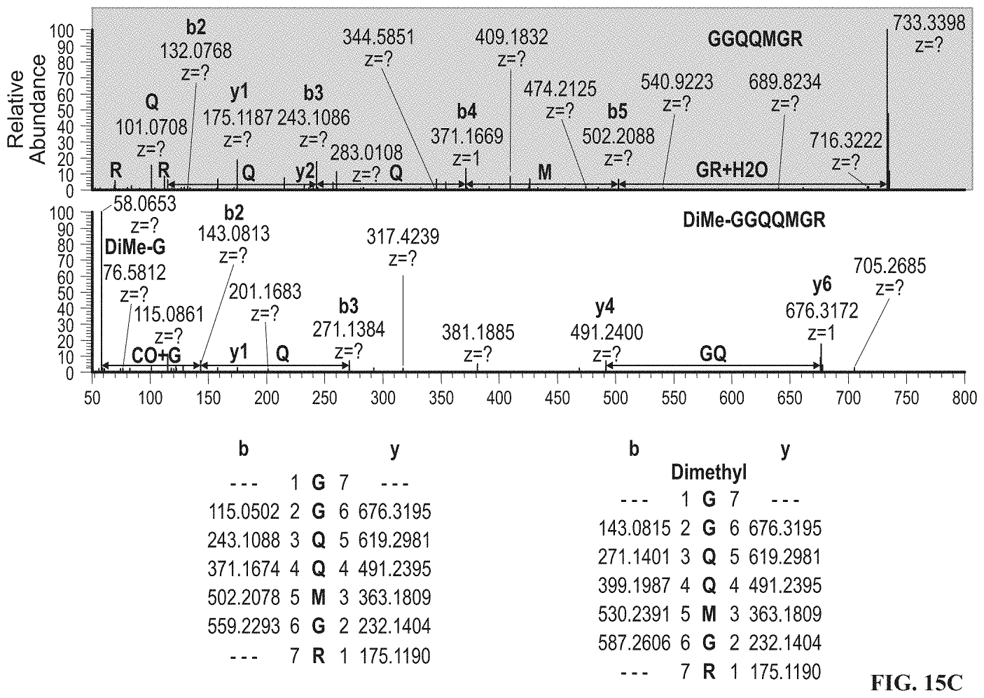

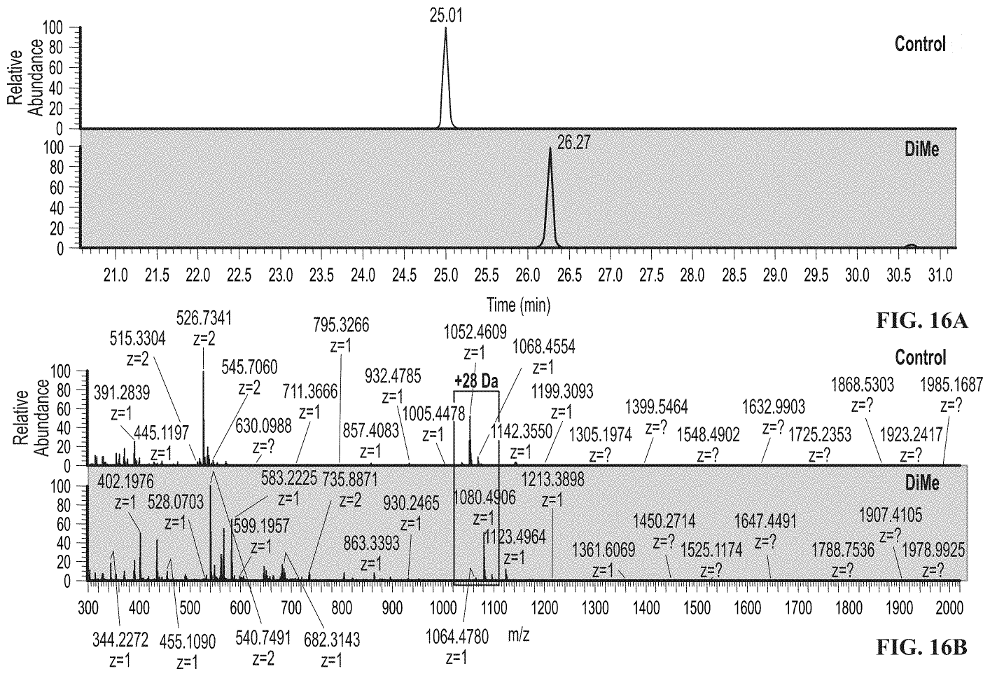

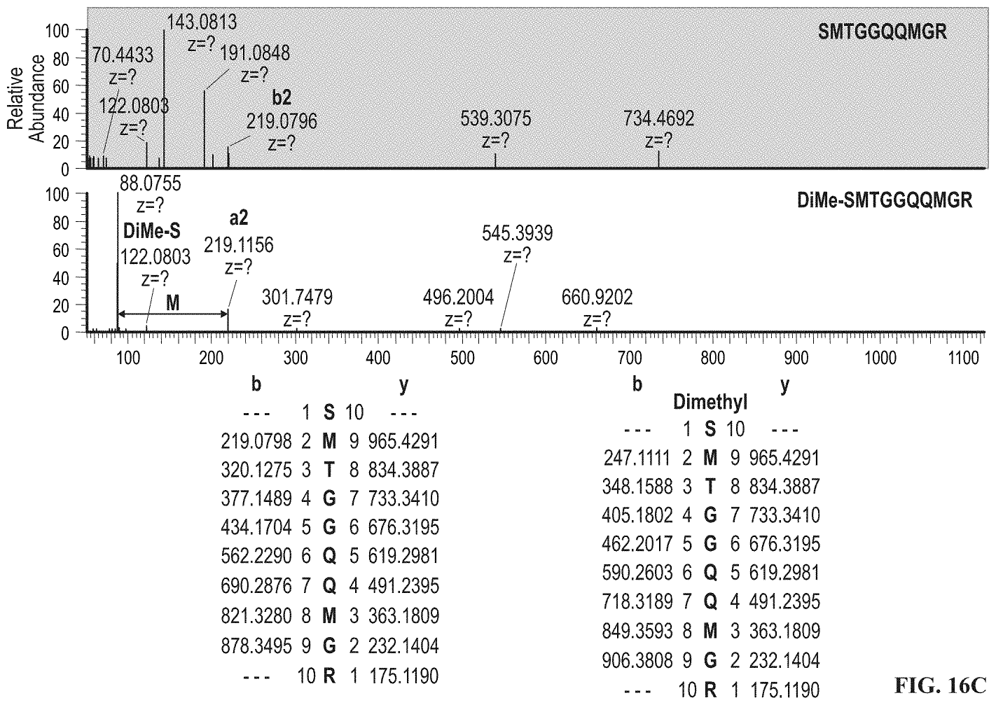

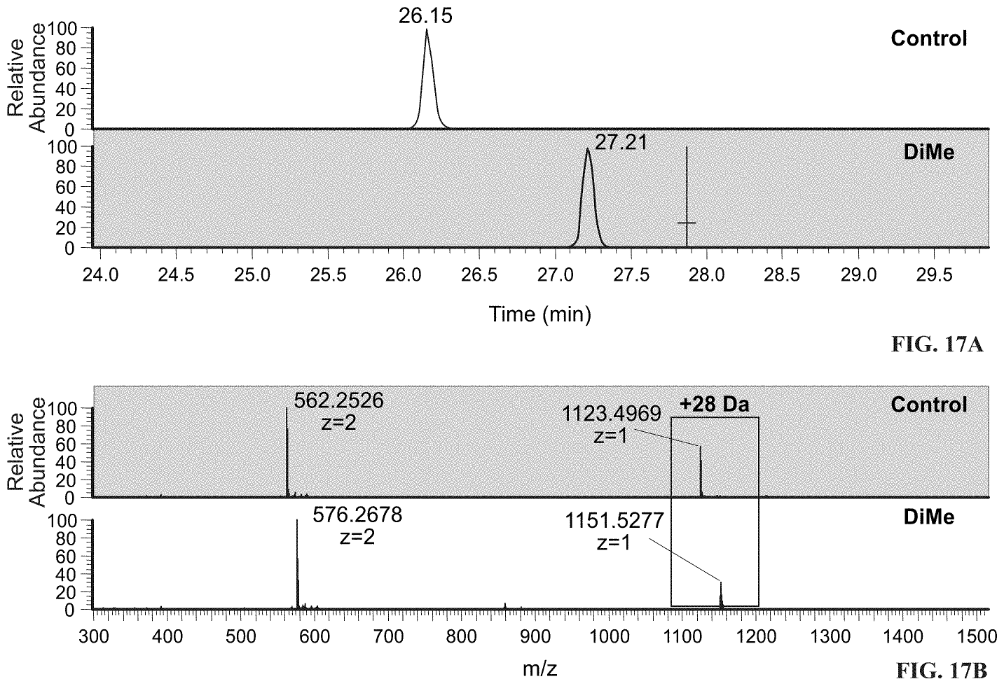

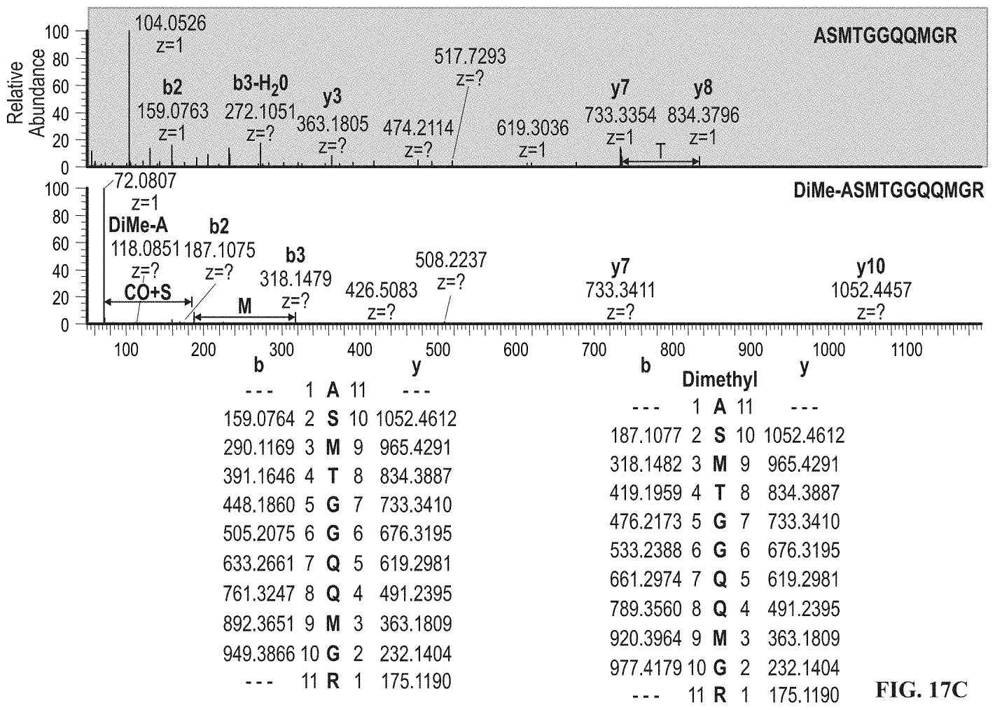

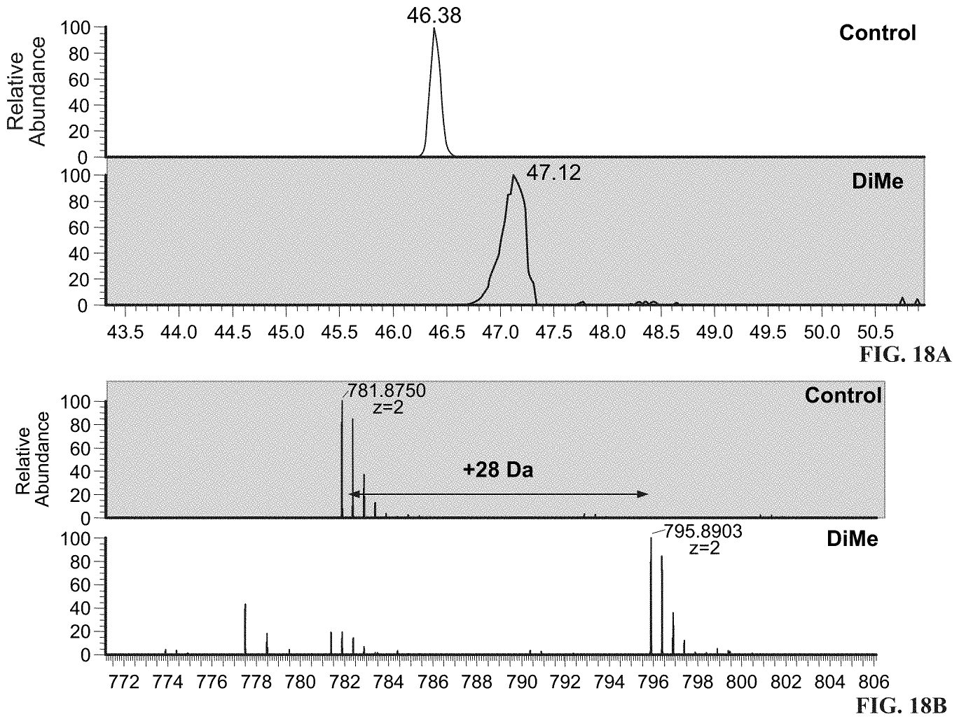

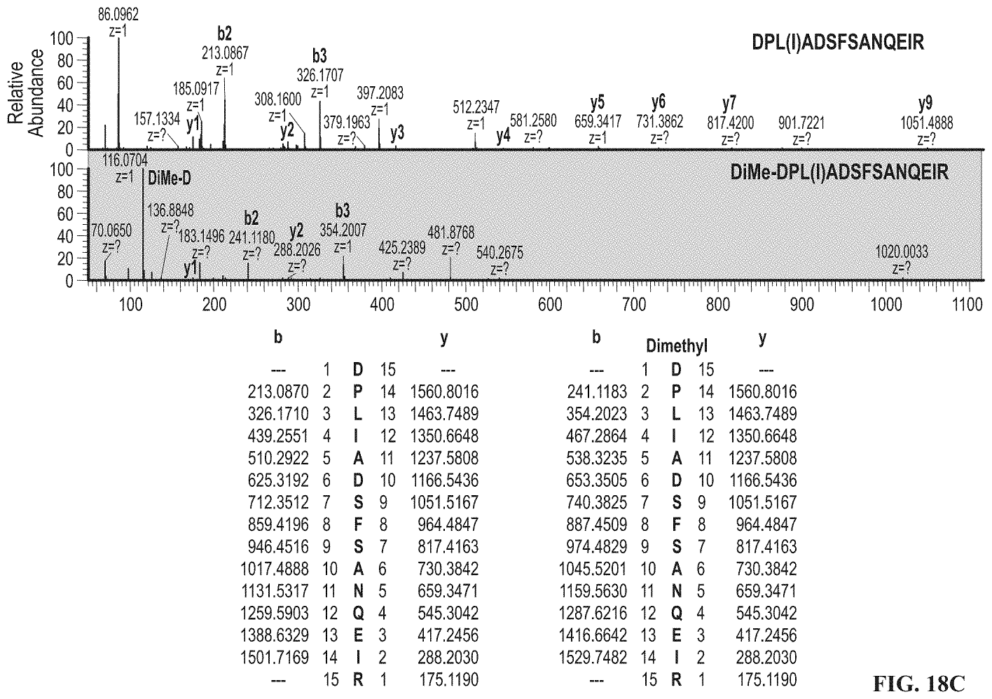

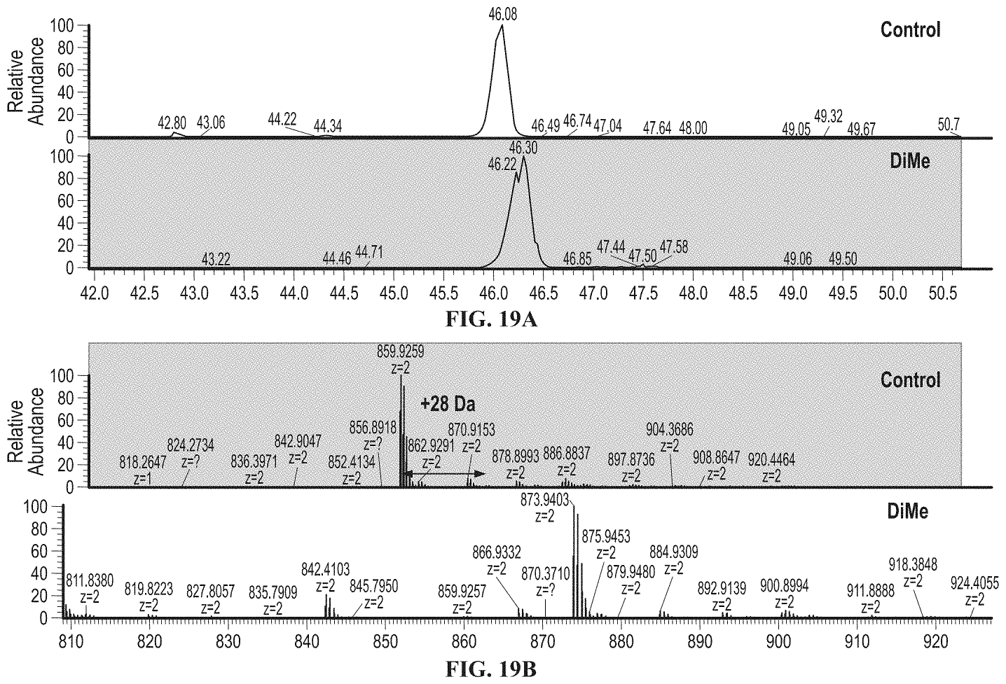

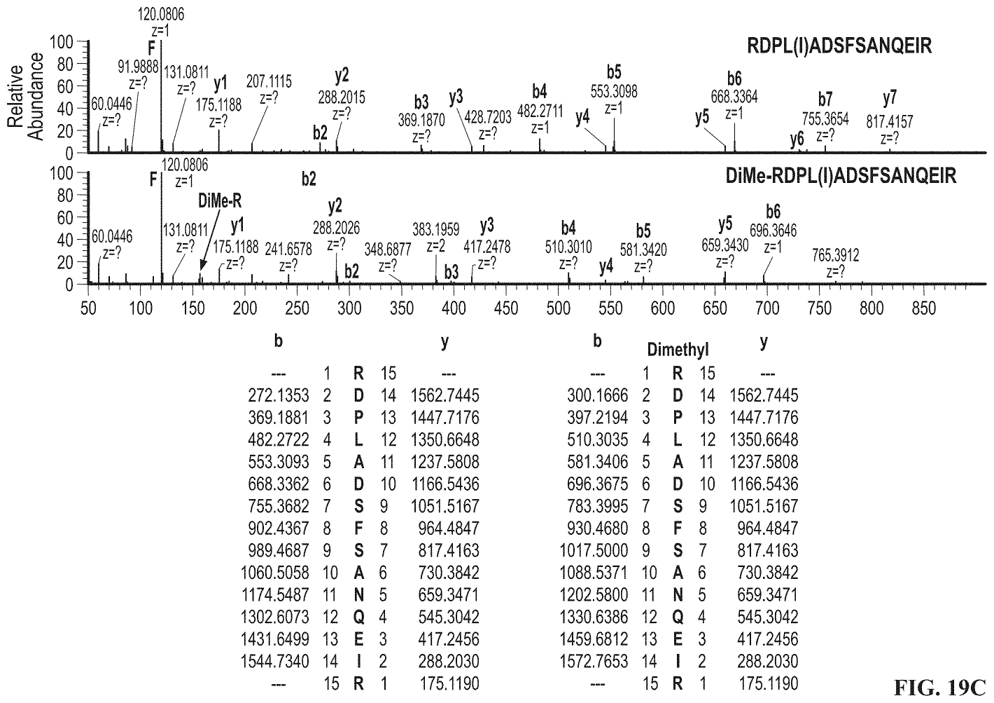

The same process was repeated for additional FabRICATOR® N-terminal peptides. As shown in FIG. 15A-C, N-terminal peptide 2 was sequenced and identified as GGQQMGR (SEQ ID NO: 3). As shown in FIG. 16A-C, N-terminal peptide 3 was sequenced and identified as SMTGGQQMGR (SEQ ID NO: 4). As shown in FIG. 17A-C, N-terminal peptide 4 was sequenced and identified as ASMTGGQQMGR (SEQ ID NO: 5). As shown in FIG. 18A-C, N-terminal peptide 5 was sequenced and identified as DPL(I)ADSFSANQEIR (SEQ ID NO: 6). As shown in FIG. 19A-C, N-terminal peptide 6 was sequenced and identified as RPDL(I)ADSFSANQEIR (SEQ ID NO: 7). In all cases, the method of the present invention allowed for efficient labeling and identification of the N-terminal peptide and the N-terminal amino acid residue, which in turn allowed for identification of b ions and subsequent amino acid sequencing.

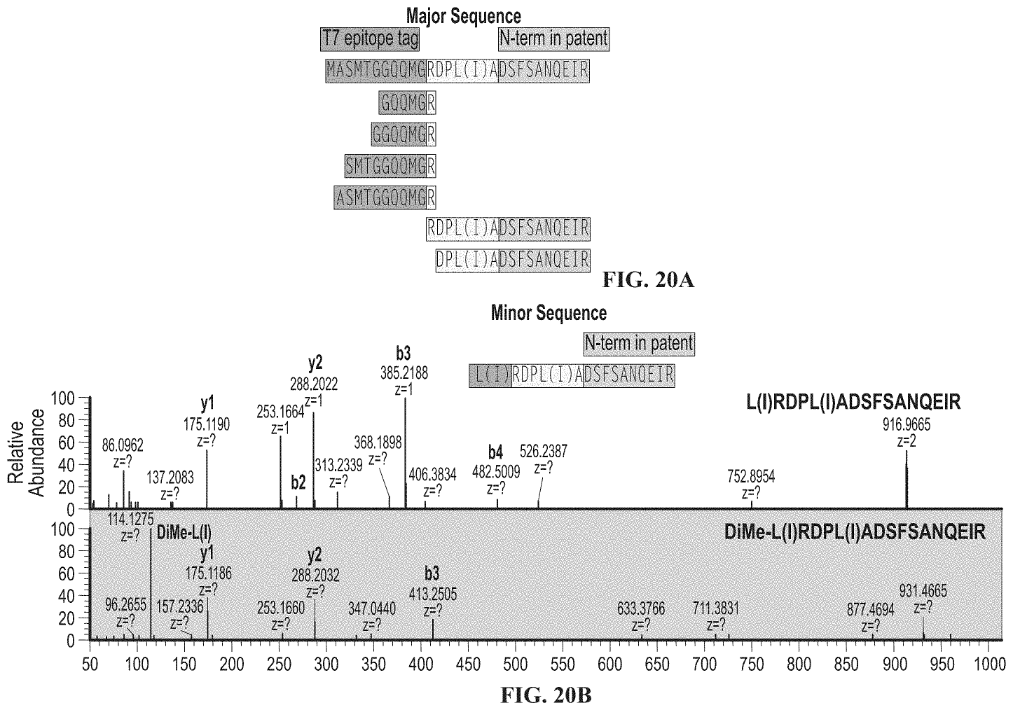

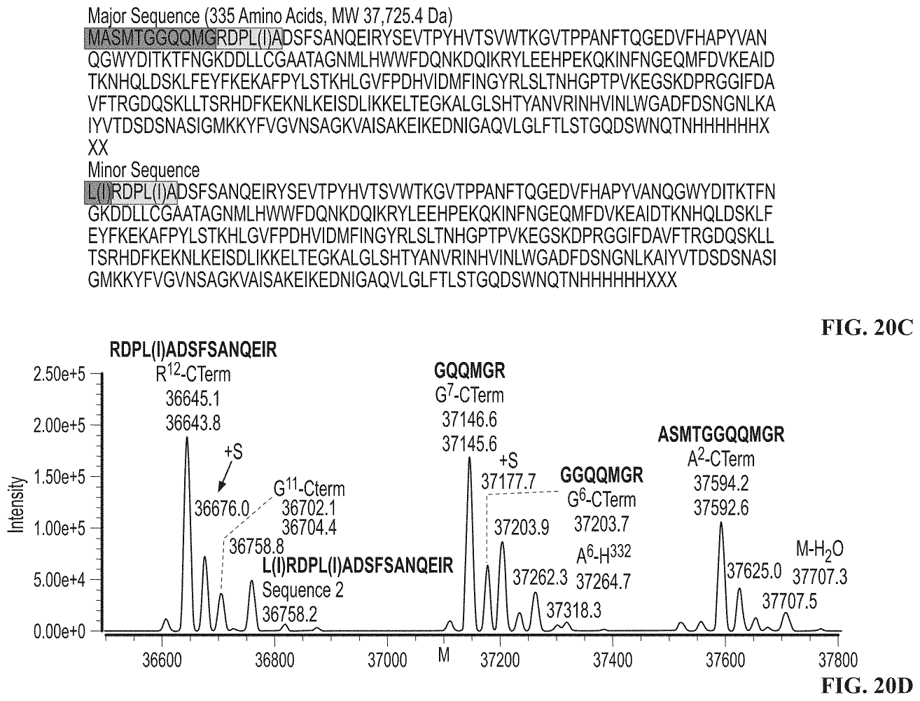

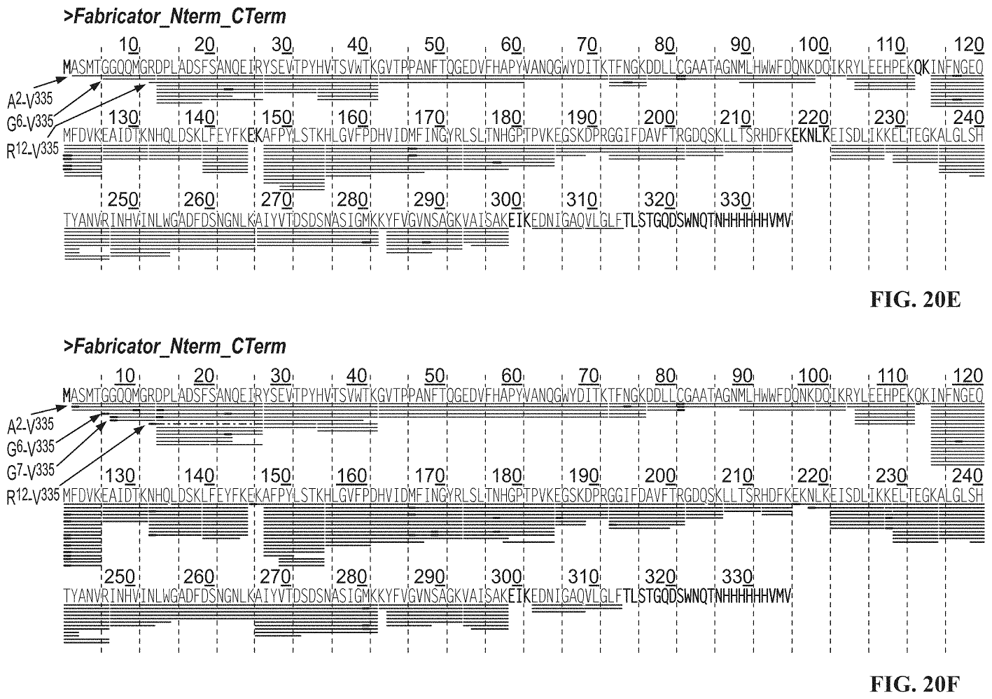

The results of sequencing the FabRICATOR® N-terminal are summarized in FIG. 20A, which shows the major N-terminal sequence as identified here and its relative position to the disclosed IdeS N-terminal sequence. The N-terminal sequence MASMTGGQQMG (SEQ ID NO: 8) was identified as the T7 epitope tag, derived from the T7 major capsid protein of the T7 gene. The T7 tag is commonly engineered onto an N-terminus or C-terminus of a protein of interest to facilitate analysis of the protein using immunochemical methods. Additionally, a minor N-terminal sequence identified using this method is depicted in FIG. 20B.

The full sequence of FabRICATOR® including the major or minor N-terminal sequences discovered herein is shown in FIG. 20C. The full FabRICATOR® sequence with the major N-terminal sequence has a predicted molecular weight of 37,725.4 Da, corresponding to the disclosed FabRICATOR® molecular weight of 37,725 Da. The identified N-terminal sequences were further validated by the use of intact mass spectrometry, with an exemplary mass spectrum shown in FIG. 20D. Various species of FabRICATOR® with total masses corresponding to the variants comprising the N-terminal sequences identified herein are annotated.

The sequence coverage of FabRICATOR® from the above analysis can be seen with a comparison of the control sample (FIG. 20E) versus the dimethylated sample (FIG. 20F). Dimethylation allowed for superior identification of N-terminal peptides compared to the control, and a clear demarcation of common truncation sites in the N-terminal T7 tag, reproducing the effectiveness of the method of the present invention in detecting truncation sites as shown in Example 4.

The method disclosed herein provides an efficient technique for de novo N-terminal sequencing with minimal added time (about 30 minutes) or difficulty when added to a conventional peptide mapping protocol. Sequencing using position-selective one-pot dimethylation significantly improved the signal intensity of N-terminal peptides, showed high labeling efficiency, allowed for the identification of truncation sites, allowed for sequencing even of predominantly blocked N-termini, differentiated between in vivo truncation sites and enzymatic digestion sites, and was shown to accurately sequence an unknown N-terminal consistent with intact mass spectrometry results.

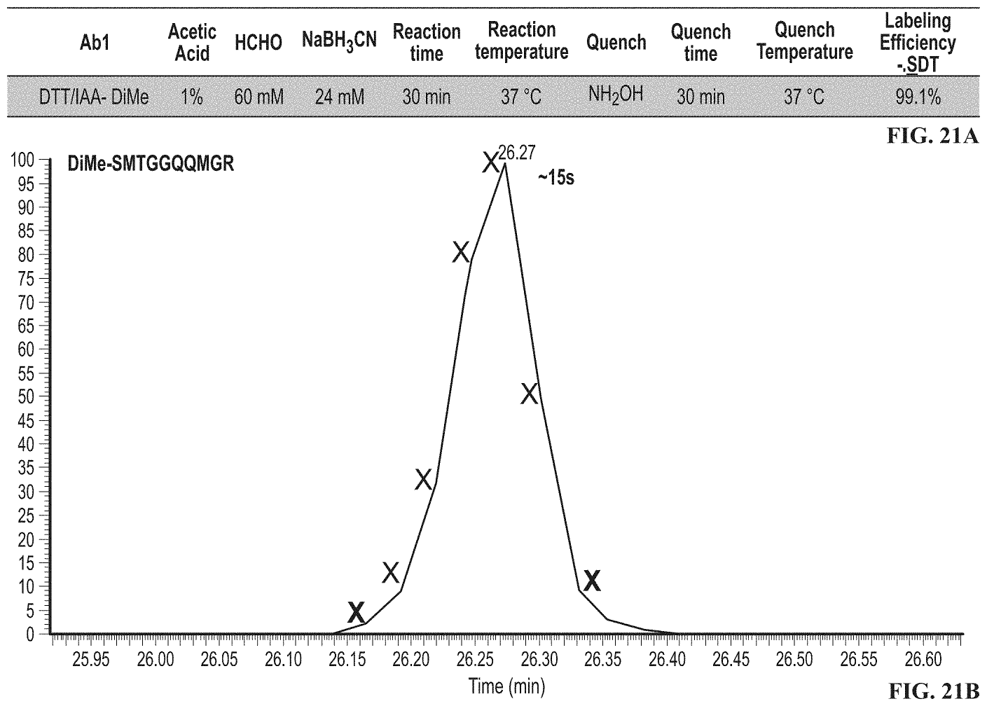

Further optimization of the method herein is contemplated. For example, labeling efficiency was further increased by using position-selective dimethylation after reduction and alkylation steps. Exemplary experimental parameters are shown in FIG. 21A (compare to FIG. 10), with a demonstrated labeling efficiency of 99.1%. This protocol is described in detail under “Further optimized protocol” above.

An additional optimization method is immonium ion-triggered MS/MS data acquisition. An immonium ion generated in HCD-MS/MS may be identified in real time by the instrument in order to identify an N-terminal sequence and tailor the fragmentation technique accordingly. Immonium-ion triggered MS/MS data acquisition could simplify data analysis. An exemplary schematic for automated identification of an immonium ion is shown in FIG. 21B.

While specific reagents, analytes, and method parameters are described as examples above, it should be understood that the method of the present invention is not limited to these examples and may be applied using a variety of reagents, analytes, or method parameters as determined by a person of skill in the art.

What is claimed is:

1. A method for determining an amino acid sequence of an N-terminal domain of a protein of interest, comprising:
(a) contacting a sample including a protein of interest to at least one dimethylation reagent to form a dimethylation mixture;
(b) contacting said dimethylation mixture to at least one quenching reagent to form a quenched mixture;
(c) subjecting said quenched mixture to liquid chromatography-mass spectrometry analysis, wherein said analysis ionizes at least one dimethylated amino acid residue to form at least one immonium ion;
(d) identifying at least one N-terminal peptide based on the presence of said at least one immonium ion; and
(e) comparing a mass spectrum of said at least one N-terminal peptide of (d) to a mass spectrum of a corresponding at least one N-terminal peptide of a non-dimethylated control sample to determine an amino acid sequence of an N-terminal domain of said protein of interest,
wherein said at least one dimethylation reagent of (a) is contacted under conditions that preferentially lead to the dimethylation of an N-terminal α-amine.

2. The method of claim 1, wherein said protein of interest is an antibody, a bispecific antibody, a monoclonal antibody, a fusion protein, an antibody-drug conjugate, an antibody fragment, or a protein pharmaceutical product.

3. The method of claim 1, wherein said at least one dimethylation reagent is selected from a group consisting of HCHO, NaBH₃CN, heavy isotopes thereof, and a combination thereof.

4. The method of claim 1, wherein said dimethylation mixture has a pH below 3.

5. The method of claim 1, wherein said dimethylation mixture includes acetic acid.

6. The method of claim 1, wherein said dimethylation mixture has a temperature between about 20° C. and about 37° C.

7. The method of claim 1, wherein said dimethylation mixture is incubated for between about 5 minutes and about 1 hour.

8. The method of claim 1, wherein said quenching reagent is selected from a group consisting of NH₃, NH₂OH, and a combination thereof.

9. The method of claim 1, wherein said quenched mixture has a temperature between about 20° C. and about 37° C.

10. The method of claim 1, wherein said quenched mixture is incubated for between about 5 minutes and about 1 hour.

11. The method of claim 1, further comprising contacting said sample and/or said quenched mixture to at least one digestive enzyme.

12. The method of claim 11, wherein said at least one digestive enzyme is selected from a group consisting of trypsin, chymotrypsin, LysC, LysN, AspN, GluC, ArgC, and a combination thereof.

13. The method of claim 1, wherein said liquid chromatography comprises reverse phase liquid chromatography, ion exchange chromatography, size exclusion chromatography, affinity chromatography, hydrophobic interaction chromatography, hydrophilic interaction chromatography, mixed-mode chromatography, or a combination thereof.

14. The method of claim 1, wherein said liquid chromatography system is coupled to said mass spectrometer.

15. The method of claim 1, wherein said mass spectrometer is an electrospray ionization mass spectrometer, nano-electrospray ionization mass spectrometer, or a triple quadrupole mass spectrometer.

16. The method of claim 1, wherein said mass spectrometer is capable performing a multiple reaction monitoring or parallel reaction monitoring.

17. The method of claim 1, further comprising contacting said sample and/or said quenched mixture to at least one alkylating agent.

18. The method of claim 17, wherein said alkylating agent is iodoacetamide.

19. The method of claim 1, further comprising contacting said sample and/or said quenched mixture to at least one reducing agent.

20. The method of claim 19, wherein said reducing agent is dithiothreitol.

21. The method of claim 1, further comprising contact said sample to at least one denaturing agent.

22. The method of claim 21, wherein said denaturing agent is urea.


SEQUENCE LISTING (47 sequences)

SEQ ID NO: 1 - protein, 10 aa, synthetic construct
DSFSANQEIR

SEQ ID NO: 2 - protein, 6 aa, synthetic construct
GQQMGR

SEQ ID NO: 3 - protein, 7 aa, synthetic construct
GGQQMGR

SEQ ID NO: 4 - protein, 10 aa, synthetic construct
SMTGGQQMGR

SEQ ID NO: 5 - protein, 11 aa, synthetic construct
ASMTGGQQMGR

SEQ ID NO: 6 - protein, 15 aa, synthetic construct
DPLIADSFSANQEIR

SEQ ID NO: 7 - protein, 16 aa, synthetic construct
RPDLIADSFSANQEIR

SEQ ID NO: 8 - protein, 11 aa, synthetic construct
MASMTGGQQMG

SEQ ID NO: 9 - protein, 13 aa, synthetic construct
  VARIANT 1..2: note = NV or VN
XXVDEVGGEALGR

SEQ ID NO: 10 - protein, 13 aa, synthetic construct
VNVDEVGGEALGR

SEQ ID NO: 11 - protein, 13 aa, synthetic construct
NVVDEVGGEALGR

SEQ ID NO: 12 - protein, 7 aa, synthetic construct
  SITE 1: note = Arginine-amide
  SITE 7: note = Carboxy-Arginine
RKKRKKR

SEQ ID NO: 13 - protein, 7 aa, synthetic construct
  SITE 1: note = Dimethyl-Arginine
  SITE 2: note = Dimethyl-lysine
  SITE 3: note = Dimethyl-lysine
  SITE 5: note = Dimethyl-lysine
  SITE 6: note = Dimethyl-lysine
  SITE 7: note = Carboxy-Arginine
RKKRKKR

SEQ ID NO: 14 - protein, 432 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 24 aa, synthetic construct
SDTGRPFVEMYSEIPEIIHMTEGR

SEQ ID NO: 16 - protein, 14 aa, synthetic construct
YSEIPEIIHMTEGR

SEQ ID NO: 17 - protein, 6 aa, synthetic construct
NYLTHR

SEQ ID NO: 18 - protein, 20 aa, synthetic construct
TIIDVVLSPSHGIELSVGEK

SEQ ID NO: 19 - protein, 5 aa, synthetic construct
QVTLR

SEQ ID NO: 20 - protein, 18 aa, synthetic construct
DIQMTQSPSTLSASVGDR

SEQ ID NO: 21 - protein, 316 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 22 - protein, 5 aa, synthetic construct
RDPLA

SEQ ID NO: 23 - protein, 7 aa, synthetic construct
LIRDPLA

SEQ ID NO: 24 - protein, 9 aa, synthetic construct
  VARIANT 1: note = Any amino acid
  VARIANT 2: note = Any amino acid
  VARIANT 3: note = Any amino acid
XXXLRDPLA

SEQ ID NO: 25 - protein, 15 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
XXXXXXXXXLRDPLA

SEQ ID NO: 26 - protein, 16 aa, synthetic construct
RDPLIADSFSANQEIR

SEQ ID NO: 27 - protein, 9 aa, synthetic construct
DSFANQEIR

SEQ ID NO: 28 - protein, 7 aa, synthetic construct
  SITE 1: note = Dimethyl-arginine
  SITE 7: note = Carboxy-Arginine
RKKRKKR

SEQ ID NO: 29 - protein, 6 aa, synthetic construct
  SITE 1: note = Dimethyl-glycine
GQQMGR

SEQ ID NO: 30 - protein, 7 aa, synthetic construct
  SITE 1: note = Dimethyl-glycine
GGQQMGR

SEQ ID NO: 31 - protein, 10 aa, synthetic construct
  SITE 1: note = Dimethyl-serine
SMTGGQQMGR

SEQ ID NO: 32 - protein, 11 aa, synthetic construct
  SITE 1: note = Dimethyl-alanine
ASMTGGQQMGR

SEQ ID NO: 33 - protein, 15 aa, synthetic construct
  SITE 1: note = Dimethyl-aspartic acid
DPLIADSFSANQEIR

SEQ ID NO: 34 - protein, 16 aa, synthetic construct
  SITE 1: note = Dimethyl-arginine
RDPLIADSFSANQEIR

SEQ ID NO: 35 - protein, 27 aa, synthetic construct
MASMTGGQQMGRDPLIADSFSANQEIR

SEQ ID NO: 36 - protein, 18 aa, synthetic construct
LIRDPLIADSFSANQEIR

SEQ ID NO: 37 - protein, 18 aa, synthetic construct
  SITE 1: note = Dimethyl-leucine
LIRDPLIADSFSANQEIR

SEQ ID NO: 38 - protein, 336 aa, synthetic construct
  VARIANT 334: note = Any amino acid
  VARIANT 335: note = Any amino acid
  VARIANT 336: note = Any amino acid
MASMTGGQQMGRDPLIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHHXXX

SEQ ID NO: 39 - protein, 327 aa, synthetic construct
  VARIANT 325: note = Any amino acid
  VARIANT 326: note = Any amino acid
  VARIANT 327: note = Any amino acid
LIRDPLIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHHXXX

SEQ ID NO: 40 - protein, 335 aa, synthetic construct
MASMTGGQQMGRDPLADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHHVMV

SEQ ID NO: 41 - protein, 25 aa, synthetic construct
  SITE 1: note = Dimethyl-Serine
SDTGRPFVEMYSEIPEIIHMTEGRE

SEQ ID NO: 42 - protein, 432 aa, synthetic construct; identical to SEQ ID NO: 14
  DISULFID 211..214: note = Interchain disulfide bond

SEQ ID NO: 43 - protein, 16 aa, synthetic construct
  SITE 2: note = Dimethyl-Tyrosine
MYSEIPEIIHMTEGRE

SEQ ID NO: 44 - protein, 8 aa, synthetic construct
  SITE 2: note = Dimethyl-Asn
  SITE 3: note = Dimethyl-Tyrosine
TNYLTHRQ

SEQ ID NO: 45 - protein, 22 aa, synthetic construct
  SITE 2: note = Dimethyl-Thr
NTIIDVVLSPSHGIELSVGEKL

SEQ ID NO: 46 - protein, 6 aa, synthetic construct
  SITE 1: note = Dimethyl-Gln
QVTLRE

SEQ ID NO: 47 - protein, 19 aa, synthetic construct
  SITE 1: note = Dimethyl-Asparagine
DIQMTQSPSTLSASVGDRV